(19) United States
(12) Patent Application Publication
(10) Pub. No.: US 2016/0362824 A1
(43) Pub. Date: Dec. 15, 2016

(54) NETTINGS, DIES, AND METHODS OF MAKING THE SAME

(71) Applicant: 3M INNOVATIVE PROPERTIES COMPANY, Saint Paul, MN (US)

(72) Inventors: Ronald W. Ausen, St. Paul, MN (US); Thomas P. Hanschen, Mendota Heights, MN (US); William J. Kopecky, Hudson, WI (US); Michelle L. Legatt, St. Paul, MN (US); Delony L. Langer-Anderson, Hugo, MN (US)

(21) Appl. No.: 15/121,705

(22) PCT Filed: Feb. 26, 2015
(86) PCT No.: PCT/US15/17719

§ 371 Date: Aug. 25, 2016

Related U.S. Application Data
(60) Provisional application No. 61/946,620, filed on Feb. 28, 2014.

(51) Int. Cl. D04H 3/05; B29C 47/06; A61F 13/02; B29C 47/14; D04H 3/007; D04H 3/16; B29C 47/00; B29C 47/04

(52) U.S. Cl. CPC D04H 3/05; B29C 47/0014; B29C 47/064; B29C 47/0021; B29C 47/0033; B29C 47/0064; B29C 47/043; B29C 47/145; D04H 3/007; D04H 3/163; A61F 13/0273; B29L 2028/00

(57) ABSTRACT

Netting comprising an array of polymeric strands, wherein the polymeric strands are periodically joined together at bond regions throughout the array, with at least two polymeric strands having a different height or color. Nettings described herein have a variety of uses, including wound care, tapes, filtration, absorbent articles, pest control articles, geotextile applications, water/vapor management in clothing, reinforcement for non-woven articles, self bulking articles, floor coverings, grip supports, athletic articles, and pattern coated adhesives.

### BACKGROUND

Polymeric nets are used for a wide variety of applications, including reinforcement of paper articles or cheap textiles (e.g., in sanitary paper articles, paper cloth, and heavy duty bags), non-woven upholstery fabrics, window curtains, decorative netting, wrapping material, mosquito netting, protective gardening netting against insects or birds, backing for growing of grass or plants, sport netting, light fishing netting, and filter materials.

Extrusion processes for making polymeric nets are well known in the art. Many of these processes require complex dies with moving parts. Many of these processes can only be used to produce relatively thick netting with relatively large diameter strands and/or relatively large mesh or opening sizes.

Polymeric netting can also be obtained from films by slitting a pattern of intermittent lines, which are mutually staggered, and expanding the slit film while stretching monoaxially or biaxially. This process tends to produce netting of a relatively large mesh and with relatively weak cross-points.

There exists a need for alternative netting compositional arrangements and the characteristics they offer, as well as methods to make the alternative netting compositional arrangements.

### SUMMARY

In one aspect, the present disclosure describes a netting comprising an array of polymeric strands (in some embodiments. at least alternating first and second (optionally third, fourth, or more) polymeric strands) periodically joined together at bond regions throughout the array, but do not substantially cross over each other (i.e., at least 50 (at least 55, 60, 65, 70, 75, 80, 85, 90, 95, 99, or even 100) percent by number), wherein the netting has a thickness up to 1000 micrometers (in some embodiments up to 750 micrometers , in some embodiments, up to 500 micrometers, 250 micrometers, 100 micrometers, 75 micrometers, 50 micrometers, or even up to 25 micrometers; in a range from 10 micrometers to 750 micrometers, 10 micrometers to 750 micrometers, 10 micrometers to 500 micrometers, 10 micrometers to 250 micrometers, 10 micrometers to 100 micrometers, 10 micrometers to 75 micrometers, 10 micrometers to 50 micrometers, or even 10 micrometers to 25 micrometers). For embodiments having at least first and second polymeric strands, the polymers of the first and second polymeric strands may be the same or different. In embodiments, the first and second polymeric strands each exhibit a different color.

In another aspect, the present disclosure provides a polymeric netting including at least two different types of generally continuous elements, one of which is ribbon like and oriented on its edge within the netting. The ribbon-like strand can be at least partially held in place by a second element: an oscillating strand. The polymeric ribbons have a major surface that is intermittently bonded to only one polymeric strand. The oscillating polymeric strands, as the description suggests, oscillate between bond region on adjacent ribbon-like strands. In some embodiments, the polymeric ribbons are elastic, the polymeric, oscillating strands are elastic, or both the polymeric ribbons and the polymeric strands are elastic.

In one aspect, the present disclosure provides a polymeric netting including polymeric ribbons and polymeric oscillating strands. Each of the polymeric ribbons and oscillating strands has a length and width, with the length being the longest dimension and the width being the shortest dimension.

In another aspect, the present disclosure provides an extrusion die. The extrusion die includes at least one cavity, a dispensing surface, and fluid passageways between the at least one cavity and the dispensing surface. The dispensing surface has an array of first dispensing orifices separated by an array of second dispensing orifices, and the first dispensing orifices, second dispensing orifices, and any other dispensing orifices are arranged in a single row across the dispensing surface. The first and second dispensing orifices each have a height and a width.

Nettings described herein have a variety of uses, including wound care and other medical applications (e.g., elastic bandage-like material, surface layer for surgical drapes and gowns, and cast padding), tapes (including for medical applications), filtration, absorbent articles (e.g., diapers and feminine hygiene products) (e.g., as a layer(s) within the articles and/or as part of an attachment system for the articles), pest control articles (e.g., mosquito nettings), geotextile applications (e.g., erosion control textiles), water/vapor management in clothing, reinforcement for nonwoven articles (e.g., paper towels), self bulking articles (e.g., for packaging) where the netting thickness is increased by stretching nettings with first strands have average first yield strength, and wherein the second strands have an average second yield strength that is different (e.g., at least 10 percent different) than the first yield strength, floor coverings (e.g., rugs and temporary mats), grip supports for tools, athletic articles, and pattern coated adhesives.

The term “ribbon” refers to longitudinally extending elements in an exemplary polymeric netting having a generally straight appearance.

A major surface of the polymeric ribbons is a surface defined by the height and the length of the ribbon.

The terms “multiple” and “a plurality” refer to more than one.

The term “elastic” refers to any material (such as a film that is 0.002 mm to 0.5 mm thick) that exhibits recovery from stretching or deformation. In some embodiments, a material may be considered to be elastic if, upon application of a stretching force, it can be stretched to a length that is at least about 25 (in some embodiments, 50) percent greater than its initial length and can recover at least 40 percent of its elongation upon release of the stretching force. “Elongation” in terms of percent refers to {(the extended length−the initial length)/the initial length} multiplied by 100.

### BRIEF DESCRIPTION OF THE DRAWINGS

FIG. 1 is a perspective view of an exemplary netting described herein having a set of strands periodically joined together at bond regions throughout an array;

FIG. 2 is a digital optical image at 50× of an exemplary netting described herein;

FIG. 3 is a cross-sectional side view of a digital optical image at 50× of an embodiment of a polymeric netting according to the present disclosure;

FIG. 4 is a plan view of an exemplary shim suited to form a repeating sequence of shims capable of forming a netting as described herein;

FIG. 5 is a plan view of another exemplary shim suited to form a repeating sequence of shims capable of forming a netting as described herein;

FIG. 6 is a plan view of another exemplary shim suited to form a repeating sequence of shims capable of forming a netting as described herein;

FIG. 7 is a plan view of another exemplary shim suited to form a repeating sequence of shims capable of forming a netting as described herein;

FIG. 8 is a plan view of another exemplary shim suited to form a repeating sequence of shims capable of forming a netting as described herein;

FIG. 9 is a plan view of another exemplary shim suited to form a repeating sequence of shims capable of forming a netting as described herein;

FIGS. 10A and 10B are a perspective views of the repeating sequence of shims of FIGS. 4, 5 and 6 in an assembled state;

FIGS. 11A and 11B are perspective views of the repeating sequence of shims of FIGS. 4, 5 and 7 in an assembled state;

FIGS. 12A and 12B are perspective views of the repeating sequence of shims of FIGS. 4, 8 and 9 in an assembled state;

FIG. 13 is an exploded perspective view of an exemplary mount suitable for an extrusion die composed of multiple repeats of the repeating sequence of shims of FIGS. 10-12;

FIG. 14 is a perspective view of the mount of FIG. 9 in an assembled state;

FIG. 15 is a graphical representation of the rate of change of hue angle at a given viewing angle of exemplary, multi-color nettings described herein;

FIG. 16 is a graphical representation of the rate of change of hue angle at a given viewing angle of exemplary nettings described herein;

### DETAILED DESCRIPTION

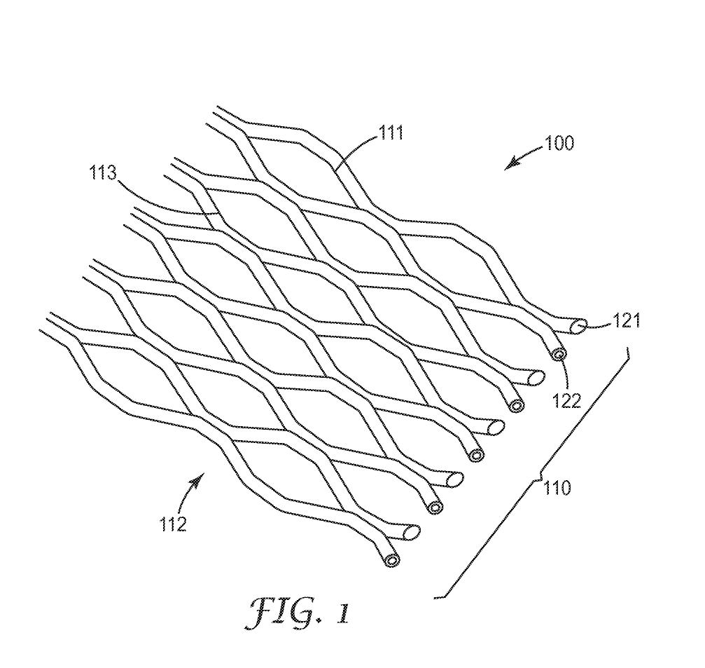

Referring to FIG. 1 an exemplary first netting 100 described herein has array of polymeric strands 110 periodically joined together at bond regions 113 throughout the array 110. Netting 100 has first and second, generally opposed major surfaces 111, 112. Bond regions 113 are generally perpendicular to first and second major surfaces 111, 112. Array 110 has first plurality of strands 121 and a second plurality of strands 122. First major surface 111 comprises the first major surfaces of the first and second plurality of strands. Second major surface 112 comprises second major surfaces of first and second plurality of strands 121, 122. The first plurality of strands 121 comprises a first material. The second plurality of strands 122 comprises a second material. The first and second materials typically differ in color or composition. Additional aspects of the netting can be found in Int. Pat. Appl. Pub. Nos. WO2013/028654, WO2013/032683, and WO2013/052371, each to Ausen et al, each incorporated by reference in their entirety herein.

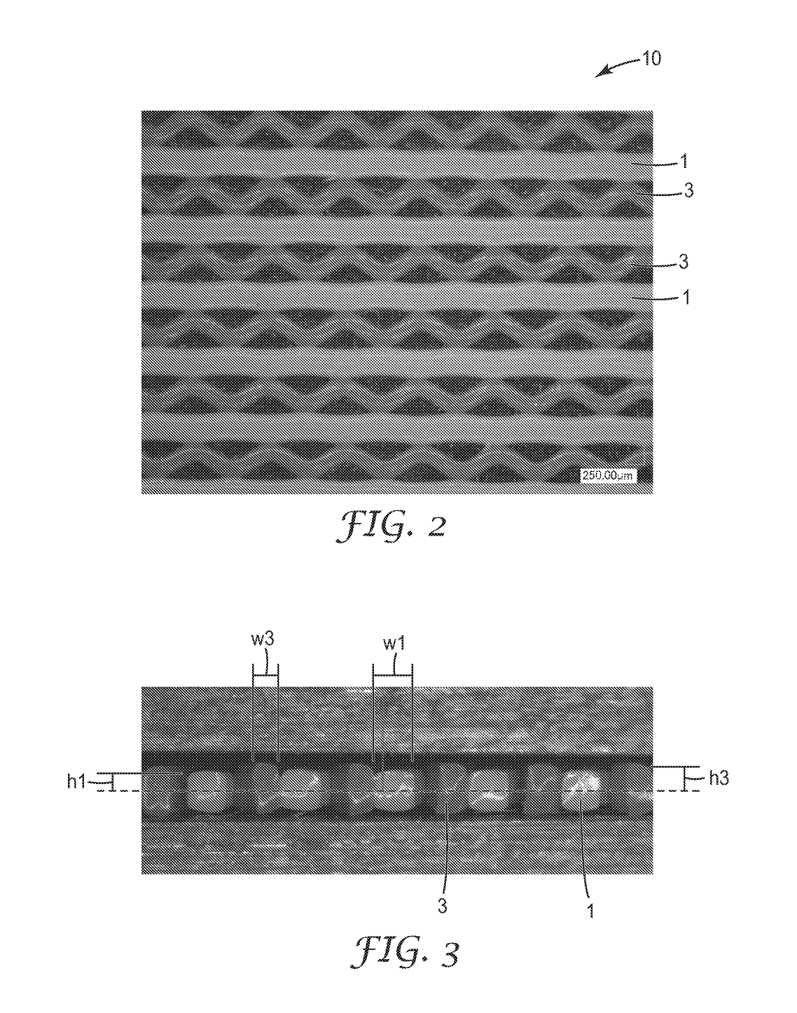

FIG. 2 is a digital image illustrating an embodiment of a polymeric netting 10 according to the present disclosure. The polymeric netting in this embodiment includes straight, ribbon-like polymeric strands 1 and oscillating polymeric strands 3. The polymeric ribbons 1 each have a first major surface that is intermittently joined to a single polymeric strand 3. When it is said that the first major surface of polymeric ribbon 1 is intermittently joined to the single polymeric strand 3, it can be observed that the polymeric strand 3 oscillates between bonding to the polymeric ribbon 1 and another portion of the netting on the opposite side of the polymeric ribbon 1. Accordingly, the polymeric strand 3 is also referred to herein as the oscillating strand. In the illustrated embodiment, two adjacent polymeric ribbons 1 are joined together by a single polymeric strand 3 at least partially alternately bonded to the two adjacent polymeric ribbons. However, this is not a requirement. Since a major surface of the polymeric ribbon 1 is intermittently bonded to a polymeric strand 3, which is at least partially alternately bonded to the polymeric ribbon 1 and another strand or ribbon of the netting, the polymeric ribbons 1 are typically not intersected by the polymeric strands 3. It should further be appreciated that any one oscillating strand 3 can be intermittently bound to another oscillating strand at the bond regions. In any embodiments of the method described above in which a net is extruded, the strands of polymer typically do not substantially cross over each other (i.e., at least 50 (at least 55, 60, 65, 70, 75, 80, 85, 90, 95, 99, or even 100) percent by number do not cross over each other).

FIG. 3 illustrates a side view of another embodiment of a polymeric netting 10 according to the present disclosure. The polymeric ribbons 1 and polymeric strands 3 each have a length, width “w1” and “w3”, and height “h1” and “h3”. The length of the polymeric ribbons 1 and strands 3 is the longest dimension and is not shown in FIG. 2. The width is the shortest dimension. The height “h1” of the ribbons and the height “h3” of the strands is typically between the length and width of each, respectively. However, the strands 3 can also have heights “h3” that are substantially the same as their widths “w3”. For circular strands, the height and width may both be referred to as diameter. The height of the polymeric strand is generally greater than that of the polymeric ribbon.

In the embodiment depicted in FIGS. 2 and 3, the polymeric ribbons and polymeric, oscillating strands alternate. In some embodiments of the polymeric netting according to the present disclosure and/or made according to the method disclosed herein, the polymeric oscillating strands alternate with at least one of the first or second polymeric ribbons in at least a portion of the polymeric netting. This means one polymeric strand is disposed between any two adjacent polymeric ribbons and one polymeric ribbon is disposed between any two adjacent polymeric strands. In other embodiments any one oscillating strand can be intermittently bound to another oscillating strand at the bond regions, such that two or more oscillating strands are joined between any two adjacent polymeric ribbon-like strands.

The shape of the individual polymeric ribbons and polymeric strands in a polymeric netting disclosed herein can depend on a variety of factors. As described above, the polymeric strands, which are typically taller than the polymeric ribbons, exit the die at a faster rate than the polymeric ribbons and are oscillating. Therefore, in some embodiments, the polymeric ribbons may be substantially straight, for example, when no extension force is placed on the polymeric netting. However, depending on the different in height between the polymeric ribbons and strands, the placement of the polymeric strands on the major surface of the polymeric ribbons, and the modulus of the materials from which the polymeric ribbons and polymeric strands are made, both the polymeric ribbons and polymeric strands may occupy a sinusoidal path in the lengthwise direction. FIG. 3, for example, illustrates a top view of a polymeric netting disclosed herein in which a portion of the polymeric ribbons appear straight, and a portion of the polymeric ribbons appear to oscillate somewhat sinusoidally.

While in FIGS. 2 and 3, the widths of the polymeric ribbons are each about the same, and the widths of the polymeric strands are all about the same, this is not a requirement. The widths of the polymeric ribbons and/or polymeric strands may change across the netting (e.g., in a direction transverse to the length of the polymeric ribbons and polymeric strands). For example, at least one of the polymeric ribbons or polymeric strands may have a larger width at the center of the netting than on the edges or vice versa.

While in FIGS. 2 and 3, the spacings between the various polymeric ribbons and polymeric strands in the polymeric netting are approximately equal, this is also not a requirement. The spacing between any two adjacent polymeric ribbons or any two adjacent polymeric strands can vary in the cross-web direction. For example, any two adjacent polymeric ribbons or any two adjacent polymeric strands may be positioned more closely together at the center of the netting than on the edges or vice versa. In a typically cross-sectional planar view, not all of the polymeric strands would appear to be identically bonded to the major surfaces of the polymeric ribbons.

Although other methods may be useful, extruding the netting includes providing an extrusion die having a plurality of shims positioned adjacent to one another, the shims together defining a first cavity and a second cavity, the extrusion die having a plurality of first dispensing orifices in fluid communication with the first cavity and having a plurality of second dispensing orifices connected to the second cavity, such that the first and second dispensing orifices are alternated. First polymeric strands are dispensed from the first dispensing orifices at a first strand speed while simultaneously second polymeric strands are dispensed from the second dispensing orifices at a second strand speed. In some embodiments, the first strand speed is in a range from 2 to 6 or from 2 to 4 times the second strand speed. The difference in strand speed can, in some embodiments, result in a measureable difference in height, as measured from a center line, between the first strand and the second strand. In other embodiments, the extrusion speeds are the same. In some embodiments, the first cavity of the extrusion die is supplied with a first polymer at a first pressure so as to dispense the first polymer from the first array at a first strand speed, the second cavity of the extrusion die is supplied with a second polymer at a second pressure so as to dispense the second polymer from the second array at a second strand speed, wherein the first strand speed is at least 2 (in some embodiments, 2 to 6, or 2 to 4) times the second strand speed. In some embodiments, the plurality of shims comprises a plurality of a repeating sequence of shims that includes a shim that provides a passageway between the first cavity and at least one of the first dispensing orifices and a shim that provides a passageway between the second cavity and the at least one of the second dispensing orifices.

In embodiments wherein the first strand speed is faster than the second strand speed, a strand including the first polymer will appear to oscillate between adjacent strands of the second polymer. In other words, the location of the bond region between a strand dispensed at the first strand speed and two strands dispensed at the second speed will alternate between adjacent straight strands along the length of the array.

For some of these embodiments, the nettings are extruded through a plurality of shims. The plurality of shims comprises a plurality of at least one repeating sequence of shims that includes shims that provide a passageway between a first and second cavity and the first dispensing orifices. In some of these embodiments, there will be additional shims that provide a passageway between the first and/or the second cavity, and/or a third (or more) cavity and second dispensing orifices. Typically, not all of the shims of dies described herein have passageways, as some may be spacer shims that provide no passageway between any cavity and a dispensing orifice. In some embodiments, there is a repeating sequence that further comprises at least one spacer shim. The number of shims providing passageway to the first dispensing orifices may be equal or unequal to the number of shims providing a passageway to the second dispensing orifices.

In some embodiments, the first dispensing orifices and the second dispensing orifices are collinear. In some embodiments, the first dispensing orifices are collinear, and the second dispensing orifices are also collinear but offset from and not collinear with the first dispensing orifices.

In some embodiments, extrusion dies described herein include a pair of end blocks for supporting the plurality of shims. In these embodiments it may be convenient for one or all of the shims to each have one or more through-holes for the passage of connectors between the pair of end blocks. Bolts disposed within such through-holes are one convenient approach for assembling the shims to the end blocks, although the ordinary artisan may perceive other alternatives for assembling the extrusion die. In some embodiments, the at least one end block has an inlet port for introduction of fluid material into one or both of the cavities.

In some embodiments, the shims will be assembled according to a plan that provides a repeating sequence of shims of diverse types. The repeating sequence can have diverse numbers of shims per repeat.

Exemplary passageway cross-sectional shapes include square, rectangular, and other quadrilateral shapes. The shape of the passageways within, for example, a repeating sequence of shims, may be identical or different. For example, in some embodiments, the shims that provide a passageway between the first cavity and a first dispensing orifice might have a flow restriction compared to the shims that provide a conduit between the second cavity and a second dispensing orifice. The width of the distal opening within, for example, a repeating sequence of shims, may be identical or different. For example, the portion of the distal opening provided by the shims that provide a conduit between the first cavity and a first dispensing orifice could be narrower than the portion of the distal opening provided by the shims that provide a conduit between the second cavity and a second dispensing orifice.

In some embodiments, the assembled shims (conveniently bolted between the end blocks) further comprise a manifold body for supporting the shims. The manifold body has at least one (or more (e.g., two or three, four, or more)) manifold therein, the manifold having an outlet. An expansion seal (e.g., made of copper or alloys thereof) is disposed so as to seal the manifold body and the shims, such that the expansion seal defines a portion of at least one of the cavities (in some embodiments, a portion of both the first and second cavities), and such that the expansion seal allows a conduit between the manifold and the cavity.

In some embodiments, with respect to extrusion dies described herein, each of the dispensing orifices of the first and the second arrays have a width, and each of the dispensing orifices of the first and the second arrays are separated by up to 2 times the width of the respective dispensing orifice.

Typically, the passageway between cavity and dispensing orifice is up to 5 mm in length. Sometimes the first array of fluid passageways has greater fluid restriction than the second array of fluid passageways.

In some embodiments, for extrusion dies described herein, each of the dispensing orifices of the first and the second arrays have a cross sectional area, and each of the dispensing orifices of the first arrays has an area different than that of the second array.

Typically, the spacing between orifices is up to 2 times the width of the orifice. The spacing between orifices is greater than the resultant diameter of the strand after extrusion. This diameter is commonly called die swell. This spacing between orifices is greater than the resultant diameter of the strand after extrusion leads to the strands repeatedly colliding with each other to form the repeating bonds of the netting. If the spacing between orifices is too great the strands will not collide with each other and will not form the netting.

The shims for dies described herein typically have thicknesses in the range from 50 micrometers to 125 micrometers, although thicknesses outside of this range may also be useful. Typically, the fluid passageways have thicknesses in a range from 50 micrometers to 750 micrometers (in some embodiments 1000 micrometers), and lengths less than 5 mm (with generally a preference for smaller lengths for decreasingly smaller passageway thicknesses), although thicknesses and lengths outside of these ranges may also be useful. For large diameter fluid passageways several smaller thickness shims may be stacked together, or single shims of the desired passageway width may be used.

The shims are tightly compressed to prevent gaps between the shims and polymer leakage. For example, 12 mm (0.5 inch) diameter bolts are typically used and tightened, at the extrusion temperature, to their recommended torque rating. Also, the shims are aligned to provide uniform extrusion out the extrusion orifice, as misalignment can lead to strands extruding at an angle out of the die which inhibits desired bonding of the net. To aid in alignment, an alignment key can be cut into the shims. Also, a vibrating table can be useful to provide a smooth surface alignment of the extrusion tip.

The size (same or different) of the strands can be adjusted, for example, by the composition of the extruded polymers, velocity of the extruded strands, and/or the orifice design (e.g., cross sectional area (e.g., height and/or width of the orifices)). For example, a first polymer orifice that is 3 times greater in area than the second polymer orifice can generate a netting with equal strand sizes despite the velocity difference between adjacent strands.

In general, it has been observed that the rate of strand bonding is proportional to the extrusion speed of the faster strand. Further, it has been observed that this bonding rate can be increased, for example, by increasing the polymer flow rate for a given orifice size, or by decreasing the orifice area for a given polymer flow rate. It has also been observed that the distance between bonds (i.e., strand pitch) is inversely proportional to the rate of strand bonding, and proportional to the speed that the netting is drawn away from the die. Thus, it is believed that the bond pitch and the netting basis weight can be independently controlled by design of the orifice cross sectional area, the takeaway speed, and the extrusion rate of the polymer. For example, relatively high basis weight nettings, with a relatively short bond pitch can be made by extruding at a relatively high polymer flow rate, with a relatively low netting takeaway speed, using a die with a relatively small strand orifice area. Additional general details for adjusting the relative speed of strands during net formation can be found, for example, in PCT Pub. No. WO 2013/028654 (Ausen et al.), published Feb. 28, 2013, the disclosure of which is incorporated herein by reference.

Typically, the polymeric strands are extruded in the direction of gravity. This enables collinear strands to collide with each other before becoming out of alignment with each other. In some embodiments, it is desirable to extrude the strands horizontally, especially when the extrusion orifices of the first and second polymer are not collinear with each other.

In practicing methods described herein, the polymeric materials might be solidified simply by cooling. This can be conveniently accomplished passively by ambient air, or actively by, for example, quenching the extruded first and second polymeric materials on a chilled surface (e.g., a chilled roll). In some embodiments, the first and/or second polymeric materials are low molecular weight polymers that need to be cross-linked to be solidified, which can be done, for example, by electromagnetic or particle radiation. In some embodiments, it is desirable to maximize the time to quenching to increase the bond strength.

Optionally, it may be desirable to stretch the as-made netting. Stretching may orientate the strands, and has been observed to increase the tensile strength properties of the netting. Stretching may also reduce the overall strand size, which may be desirable for applications which benefit from a relatively low basis weight. As an additional example, if the materials and the degree of stretch, are chosen correctly, the stretch can cause some of the strands to yield while others do not, tending to form loft (e.g., the loft may be created because of the length difference between adjacent bonded netting strands or by curling of the bonds due to the yield properties of the strands forming the bond). The attribute can be useful for packaging applications where the material can be shipped to package assembly in a relatively dense form, and then lofted, on location. The loftiness attribute can also be useful as the loop for hook and loop attachment systems, wherein the loft created with strands enables hook attachment to the netting strands. As a second additional example, if the materials of the first and second sets of strands are of different strength, cross-machine direction stretching can cause one strand to stretch and the second set of strand to not stretch. This can be useful to create for example, elastic strands which provide machine direction elasticity, which are connected to small, oriented strands, which purpose is to hold the elastic strands in place. In some embodiments, netting could be made with cross-direction elasticity with relatively small strands that are elastic, connected to relatively large strands that are inelastic.

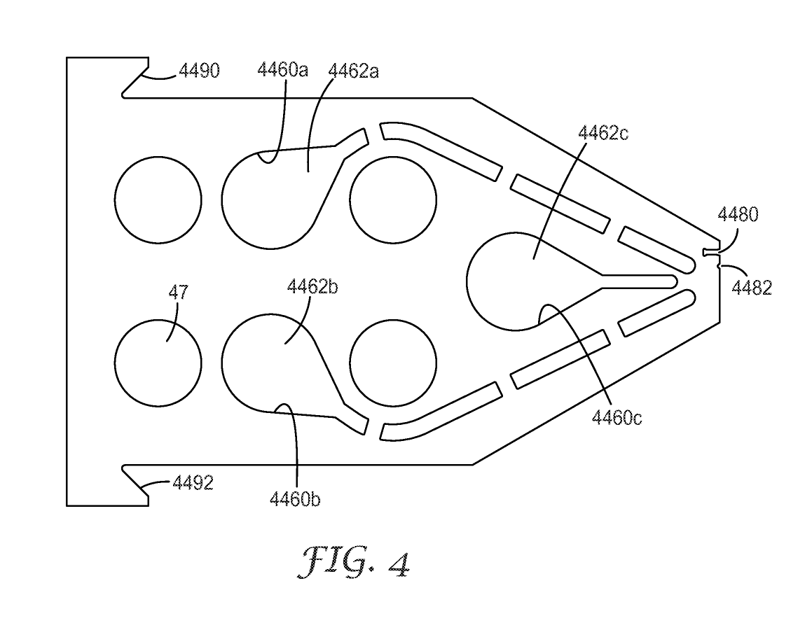

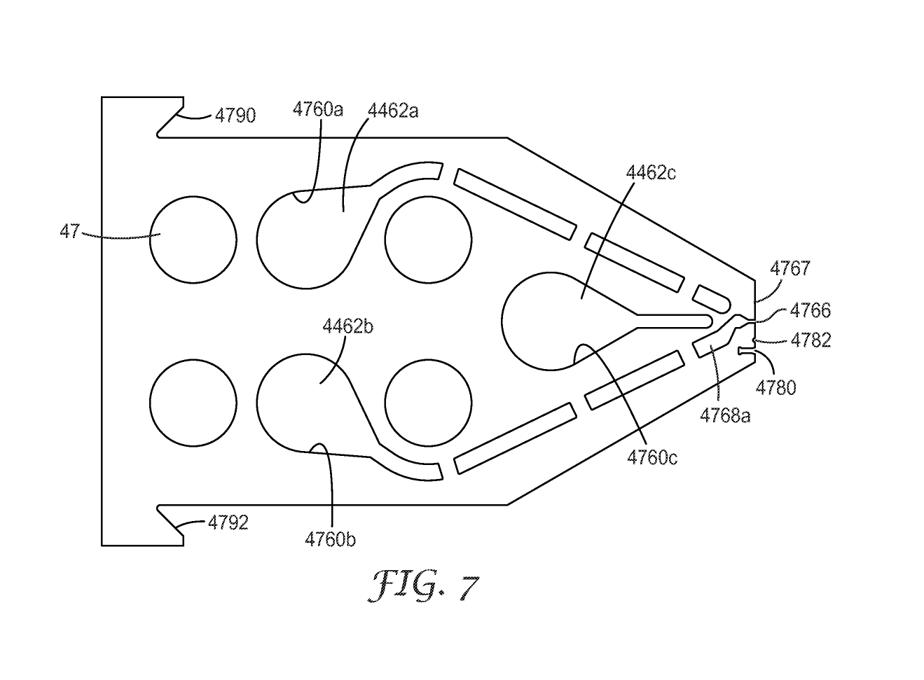

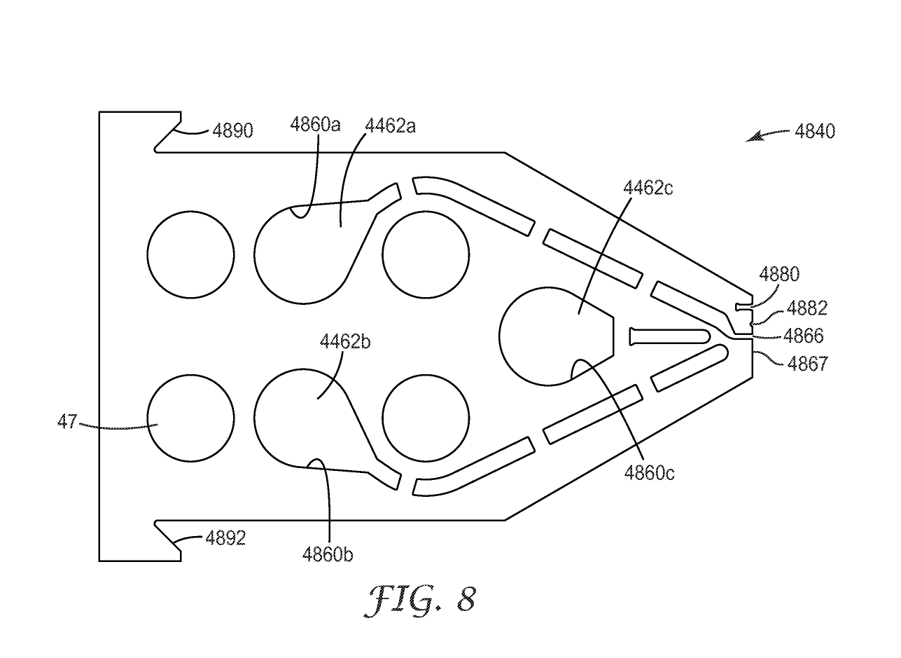

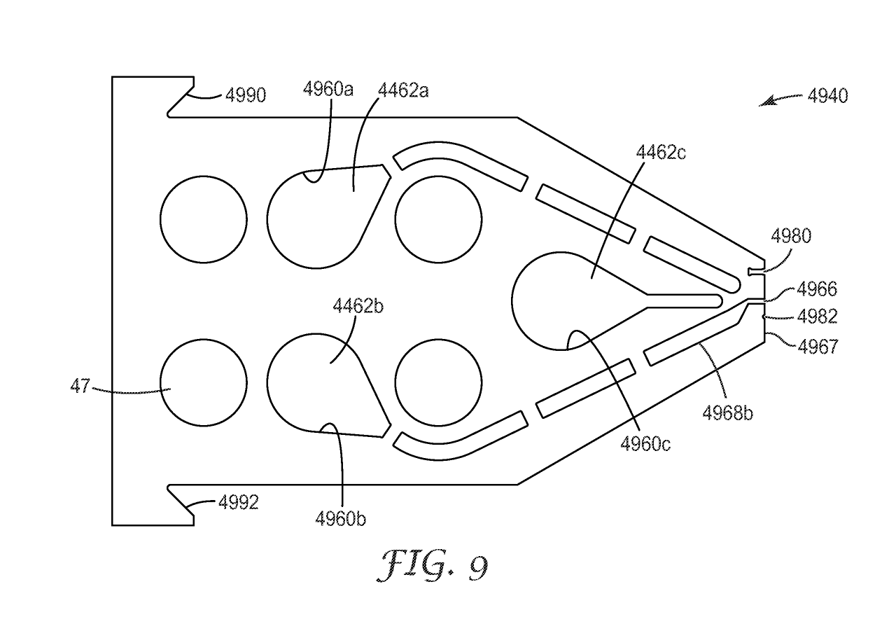

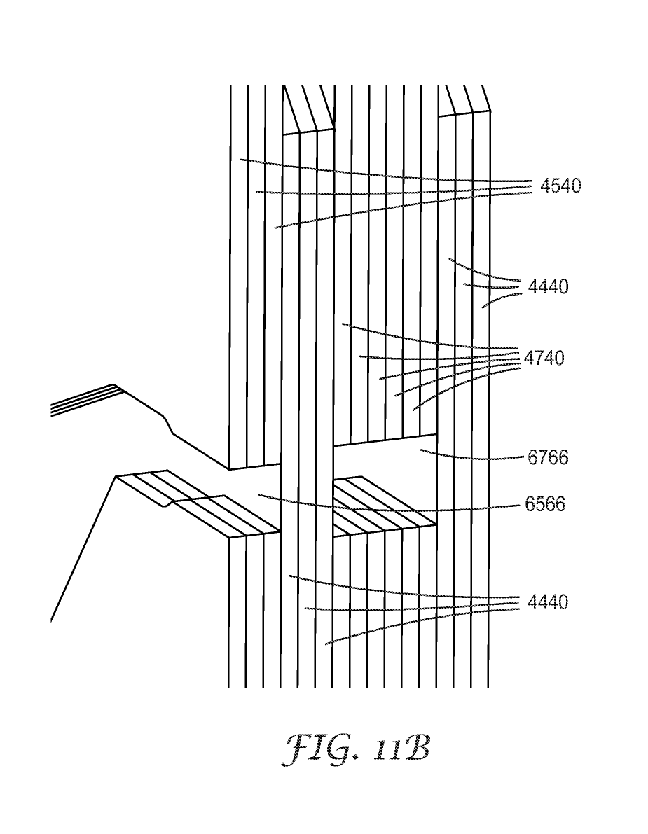

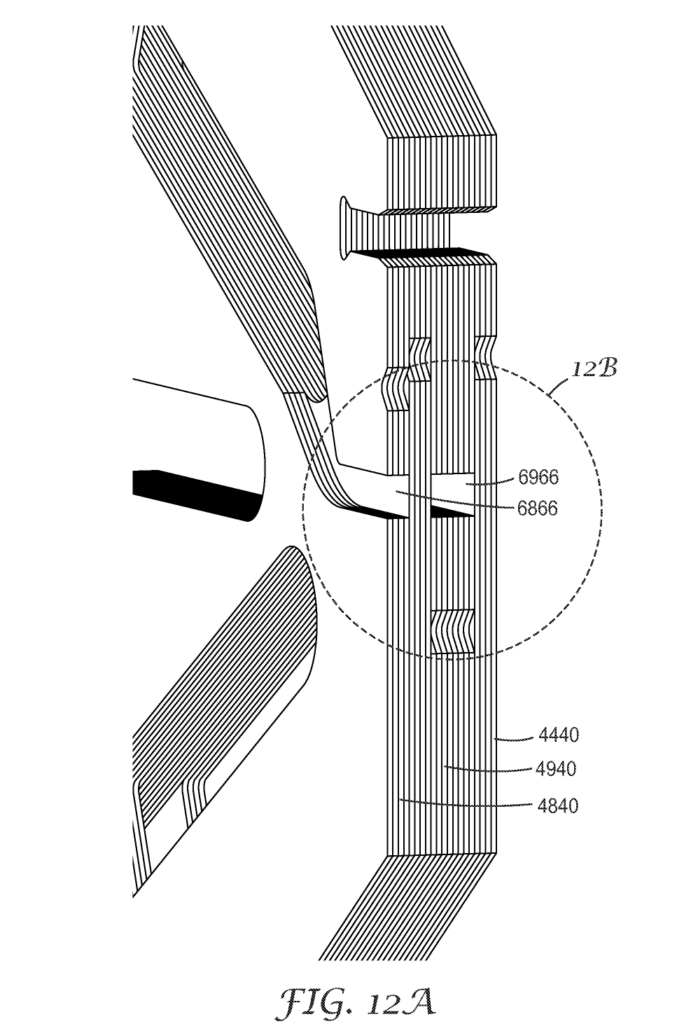

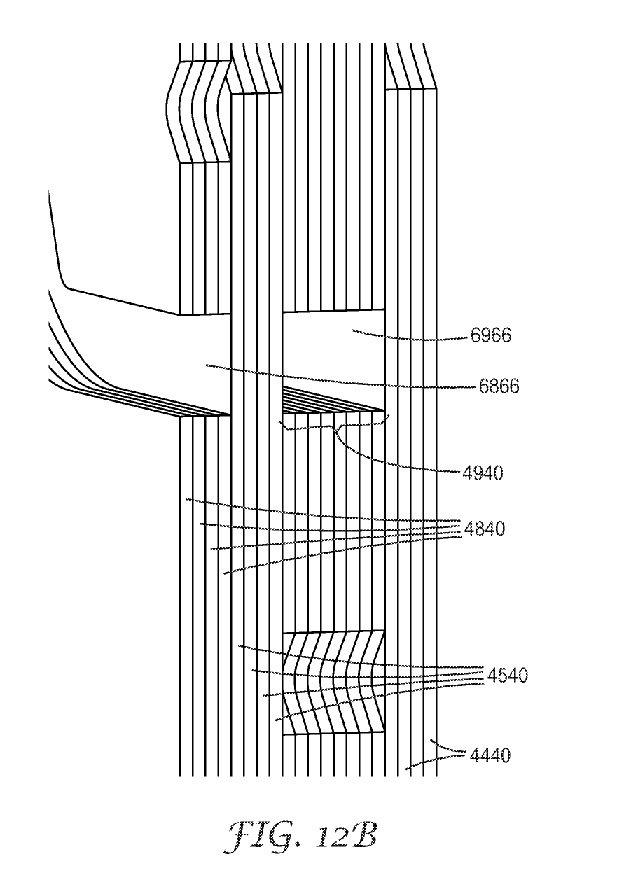

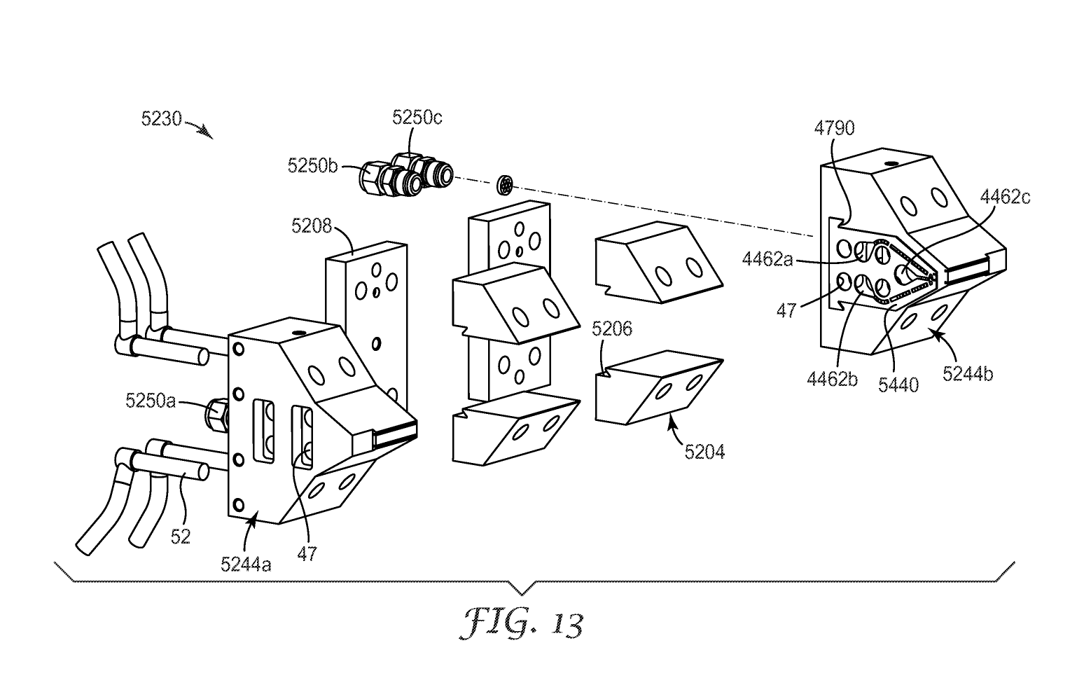

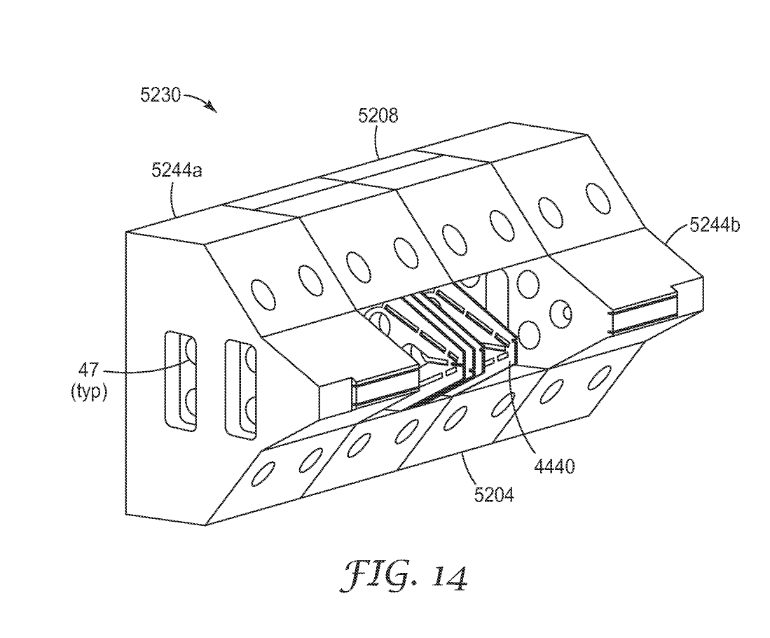

Dies and methods described herein can be used to form netting where polymeric strands and ribbons are formed of two different materials. FIGS. 4-6 illustrate exemplary shims useful for assembling an extrusion die capable of producing a netting that exhibits a color shift as described herein. FIG. 7 is a perspective view of the repeating sequence of the exemplary shims of FIGS. 4-6 in an assembled state. FIGS. 8-10 illustrate additional, exemplary shims useful for assembling an extrusion die capable of producing a netting that exhibits a color shift as described herein. FIG. 11 is a perspective view of the repeating sequence of the exemplary shims of FIGS. 8-10 in an assembled state. FIG. 12 is an exploded perspective view of a mount suitable for an extrusion die composed of multiple repeats of the repeating sequence of shims of FIGS. 4 and 8. FIG. 13 shows the mount of FIG. 12 in an assembled state.

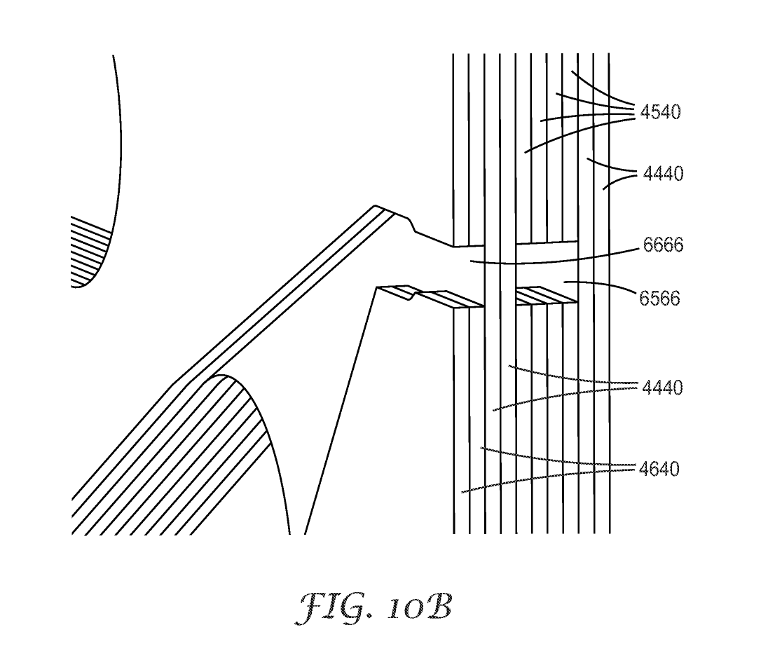

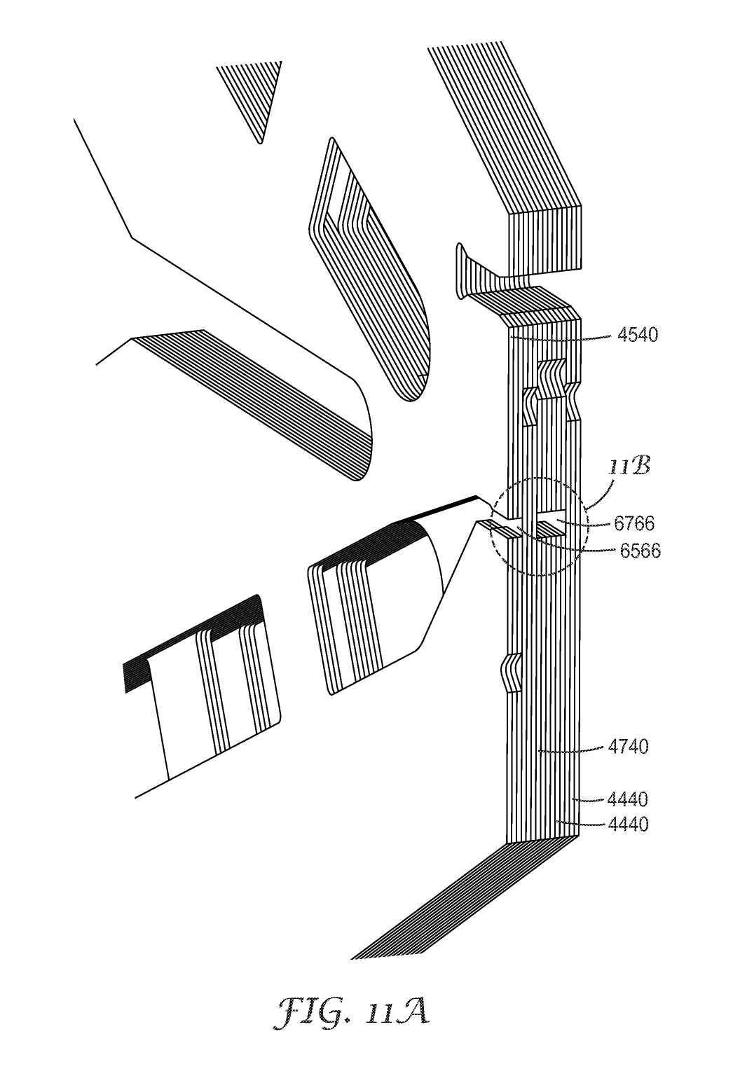

Referring now to FIG. 4, a plan view of shim 4440 from FIGS. 10-12 is illustrated. Shim 4440 has first aperture, 4460a, second aperture 4460b, and third aperture 4460c. When shim 4440 is assembled with others as shown in FIG. 10, aperture 4460a will help define first cavity 4462a, aperture 4460b will help define second cavity 4462b, and aperture 4460c will help define third cavity 4462c.

Shim 4440 has several holes 47 to allow the passage of, for example, bolts to hold shim 4440 and others to be described below into an assembly. Shim 4440 has dispensing surface 4467, and in this particular embodiment, dispensing surface 4467 has indexing groove 4480 which can receive an appropriately shaped key to ease assembling diverse shims into a die. The shim may also have identification notch 4482 to help verify that the die has been assembled in the desired manner. This embodiment has shoulders 4490 and 4492, which these can assist in mounting the assembled die in a manner which will be made clear below in connection with FIG. 13-14.

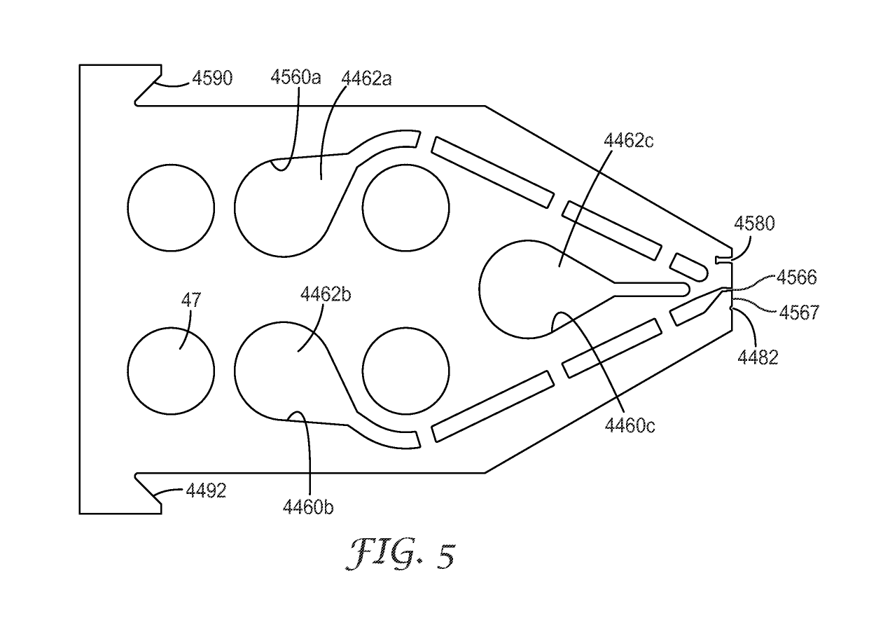

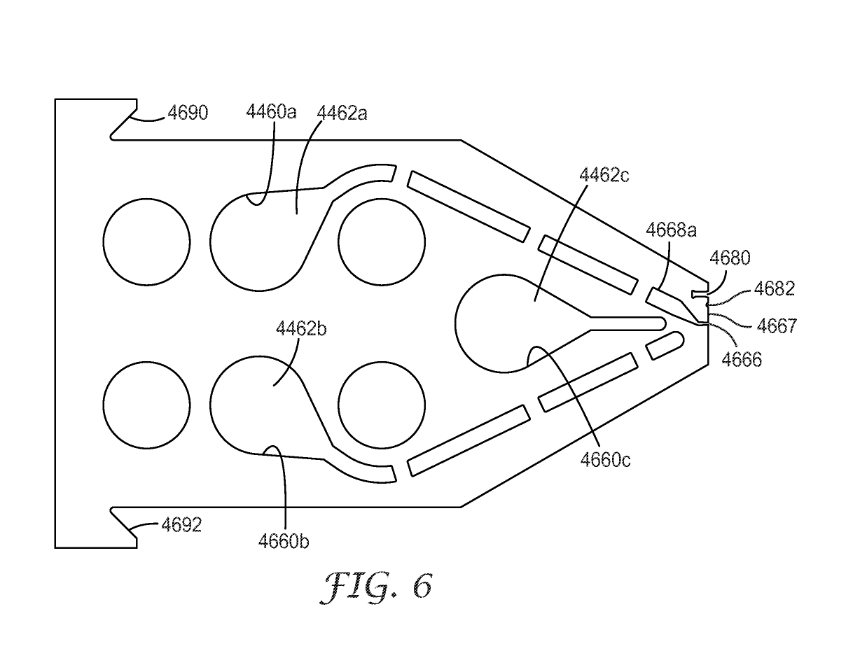

Referring now to FIG. 5, a plan view of a shim 4540 is illustrated. Shim 4540 has first aperture 4560a, second aperture 4560b, and third aperture 4560c. When shim 4540 is assembled with others as shown in FIG. 6, aperture 4560a will help define first cavity 4462a, aperture 4560b will help define second cavity 4462b, and aperture 4560c will help define third cavity 4462c. Analogous to shim 4440, shim 4540 has dispensing surface 4567, and in this particular embodiment, dispensing surface 4567 has indexing groove 4580, identification notch 4582, and shoulders 4590 and 4592. It might seem that there is no path from cavity 4462b to dispensing orifice 4566, via, for example, passageway 4568b, but this is an illusion—the flow has a route in the perpendicular-to-the-plane-of-the-drawing dimension when the repeating sequence of FIGS. 10 and 11 are completely assembled.

Referring now to FIG. 6, a plan view of shim 4640 is illustrated. Shim 4640 has first aperture 4660a, second aperture 4660b, and third aperture 4660c. When shim 4640 is assembled with others as shown in FIG. 6, aperture 4660a will help define first cavity 4462a, aperture 4660b will help define second cavity 4462b, and aperture 4660c will help define third cavity 4462c. Analogous to shim 4440, shim 4640 has dispensing surface 4667, and in this particular embodiment, dispensing surface 4667 has indexing groove 4680, an identification notch 4682, and shoulders 4690 and 4692. It might seem that there is no path from cavity 4462a to dispensing orifice 4666, via, for example, passageway 4668a, but this is an illusion—the flow has a route in the perpendicular-to-the-plane-of-the-drawing dimension when the repeating sequence of FIG. 10 is completely assembled.

Referring now to FIG. 7, a plan view of shim 4740 is illustrated. Shim 4740 has first aperture 4760a, second aperture 4760b, and third aperture 4760c. When shim 4740 is assembled with others as shown in FIG. 11, aperture 4760a will help define first cavity 4462a, aperture 4760b will help define second cavity 4462b, and aperture 4760c will help define third cavity 4462c. Analogous to shim 4440, shim 4740 has dispensing surface 4767, and in this particular embodiment, dispensing surface 4767 has indexing groove 4780, an identification notch 4782, and shoulders 4690 and 4692. It might seem that there is no path from cavity 4462a to dispensing orifice 4666, via, for example, passageway 4768a, but this is an illusion—the flow has a route in the perpendicular-to-the-plane-of-the-drawing dimension when the repeating sequence of FIG. 10 is completely assembled.

Referring now to FIG. 8, a plan view of shim 4840 is illustrated. Shim 4840 has first aperture, 4860a, second aperture 4860b, and third aperture 4860c. When shim 4840 is assembled with others as shown in FIG. 12, aperture 4860a will help define first cavity 4462a, aperture 4860b will help define second cavity 4462b, and aperture 4860c will help define third cavity 4462c. Analogous to shim 4440, shim 4840 has dispensing surface 4867, and in this particular embodiment, dispensing surface 4867 has indexing groove 4880, identification notch 4882, and shoulders 4890 and 4892. It might seem that there is no path from cavity 4462a to dispensing orifice 4866, via, for example, passageway 4868a, but this is an illusion—the flow has a route in the perpendicular-to-the-plane-of-the-drawing dimension when the repeating sequence of FIG. 12 is completely assembled.

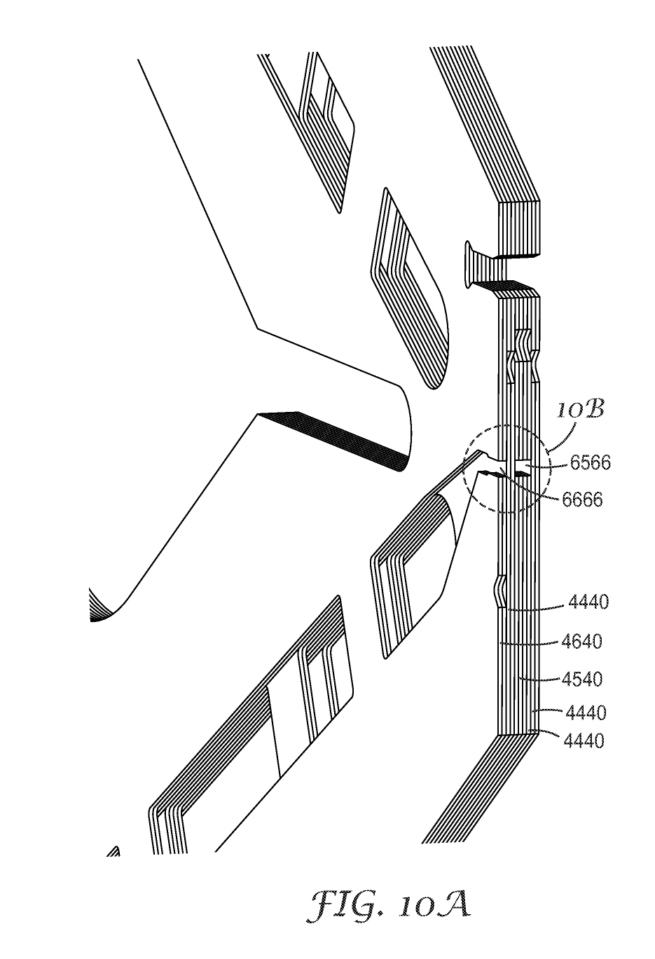

Referring now to FIG. 9, a plan view of shim 4940 is illustrated. Shim 4940 has first aperture, 4960a, second aperture 4960b, and third aperture 4960c. When shim 4940 is assembled with others as shown in FIG. 12, aperture 4960a will help define first cavity 4462a, aperture 4960b will help define second cavity 4462b, and aperture 4960c will help define third cavity 4462c. Analogous to shim 4440, shim 4940 has dispensing surface 4967, and in this particular embodiment, dispensing surface 4967 has indexing groove 4980, identification notch 4982, and shoulders 4990 and 4992. It might seem that there is no path from cavity 4462a to dispensing orifice 4966, via, for example, passageway 4968b, but this is an illusion—the flow has a route in the perpendicular-to-the-plane-of-the-drawing dimension when the repeating sequence of FIG. 12 is completely assembled.

Referring now to FIGS. 10A and 10B, a perspective assembly drawing of a repeating sequence of shims employing the shims of FIGS. 4-6 is illustrated in an assembled state. In the particular illustrated embodiment, the repeating sequence includes, from right to left as the drawing is oriented, two instances of shim 4640, two instances of shim 4440, four instances of shim 4540, and two instances of shim 4440. In this view, it is easier to appreciate how the polymer strand emerges from the egress 6566 provided by four dispensing orifices 4566 of the four instances of shim 4540 and the egress 6666 provided by the two dispensing orifices 4666 of shim 4640.

Referring now to FIGS. 11A and 11B, a perspective assembly drawing of a repeating sequence of shims employing the shims of FIGS. 4, 5 and 7 is illustrated in an assembled state. In the particular illustrated embodiment, the repeating sequence includes, from right to left as the drawing is oriented, three instances of shim 4540, three instances of shim 4440, six instances of shim 4740, and three instances of shim 4440. In this view, it is easier to appreciate how a polymer strand emerges from the egress 6766 provided by six dispensing orifices 4766 of the six instances of shim 4740 and the egress 6566 provided by three dispensing orifices 4566 of the three instances of shim 4540.

Referring now to FIGS. 12A and 12B, a perspective assembly drawing of a repeating sequence of shims employing the shims of FIGS. 4, 8 and 9 is illustrated in an assembled state. In the particular illustrated embodiment, the repeating sequence includes, from right to left as the drawing is oriented, four instances of shim 4840, four instances of shim 4440, eight instances of shim 4940, and four instances of shim 4440. In this view, it is easier to appreciate how a polymer strand emerges from the egress 6966 provided by eight dispensing orifices 4966 of the eight instances of shim 4940 and the egress 6866 provided by the four dispensing orifices 4866 of the four instances of shim 4840.

Referring now to FIG. 13, an exploded perspective view of a mount 5230 suitable for an extrusion die composed of multiple repeats of the repeating sequence of shims of FIG. 10 is illustrated, though the mount 5230 may be used for the assembled shims of FIGS. 11 and 12. Mount 5230 is particularly adapted to use combinations of shims 4440, 4540, 4640, 4740, 4840, 4940 as shown in FIGS. 4 through 9. The multiple repeats of the repeating sequence of shims of FIG. 10 are compressed between two end blocks 5244a and 5244b. Conveniently, through bolts can be used to assemble the shims to the end blocks 5244a and 5244b, passing through holes 47 in shims 4440 et al.

In this embodiment, inlet fittings 5250a and 5250b, and 5250c provide a flow path for three streams of molten polymer through end blocks 5244a and 5244b to cavities 4462a, 4462b, and 4462c. Compression blocks 5204 have a notch 5206 that conveniently engages the shoulders on the shims (e.g., 4790 and 4792 on 4740). When mount 5230 is completely assembled, compression blocks 5204 are attached by, e.g., machine bolts to backplates 5208. Holes are conveniently provided in the assembly for the insertion of cartridge heaters 52.

Referring now to FIG. 14, a perspective view of mount 5230 of FIG. 13 is illustrated in a partially assembled state. A few shims (e.g., 4440) are in their assembled positions to show how they fit within mount 5230, but most of the shims that would make up an assembled die have been omitted for visual clarity.

This sequence can make a polymeric netting in which a polymeric strand oscillates between bonding to a first and second polymeric ribbon, or adjacent oscillating strands.

Portions of the exteriors of the first and second strands (e.g., a major surface of the ribbon-like strand) bond together at the bond regions. In methods described herein for making nettings described herein, the bonding occurs in a relatively short period of time (typically less than 1 second). The bond regions, as well as the strands typically cool through air and natural convection and/or radiation. In selecting polymers for the strands, in some embodiments, it may be desirable to select polymers of bonding strands that have dipole interactions (or H-bonds) or covalent bonds. Bonding between strands has been observed to be improved by increasing the time that the strands are molten to enable more interaction between polymers. Bonding of polymers has generally been observed to be improved by reducing the molecular weight of at least one polymer and or introducing an additional co-monomer to improve polymer interaction and/or reduce the rate or amount of crystallization. In some embodiments, the bond strength is greater than the strength of the strands forming the bond. In some embodiments, it may be desirable for the bonds to break and thus the bonds will be weaker than the strands.

Suitable polymeric materials for extrusion from dies described herein, methods described herein, and nettings described herein include thermoplastic resins comprising polyolefins (e.g., polypropylene and polyethylene), polyvinyl chloride, polystyrene, nylons, polyesters (e.g., polyethylene terephthalate) and copolymers and blends thereof. Suitable polymeric materials for extrusion from dies described herein, methods described herein, and for nettings described herein also include elastomeric materials (e.g., ABA block copolymers, polyurethanes, polyolefin elastomers, polyurethane elastomers, metallocene polyolefin elastomers, polyamide elastomers, ethylene vinyl acetate elastomers, and polyester elastomers). Exemplary adhesives for extrusion from dies described herein, methods described herein, and for composite layers described herein include acrylate copolymer pressure sensitive adhesives, rubber based adhesives (e.g., those based on natural rubber, polyisobutylene, polybutadiene, butyl rubbers, styrene block copolymer rubbers, etc.), adhesives based on silicone polyureas or silicone polyoxamides, polyurethane type adhesives, and poly(vinyl ethyl ether), and copolymers or blends of these. Other desirable materials include, for example, styrene-acrylonitrile, cellulose acetate butyrate, cellulose acetate propionate, cellulose triacetate, polyether sulfone, polymethyl methacrylate, polyurethane, polyester, polycarbonate, polyvinyl chloride, polystyrene, polyethylene naphthalate, copolymers or blends based on naphthalene dicarboxylic acids, polyolefins, polyimides, mixtures and/or combinations thereof. Exemplary release materials for extrusion from dies described herein, methods described herein, and for composite layers described herein include silicone-grafted polyolefins such as those described in U.S. Pat. No. 6,465,107 (Kelly) and U.S. Pat. No. 3,471,588 (Kanner et al.), silicone block copolymers such as those described in PCT Publication No. WO96039349, published Dec. 12, 1996, low density polyolefin materials such as those described in U.S. Pat. No. 6,228,449 (Meyer), U.S. Pat. No. 6,348,249 (Meyer), and U.S. Pat. No. 5,948,517 (Meyer), the disclosures of which are incorporated herein by reference.

Many types of thermoplastic elastomers are commercially available, including those from BASF, Florham Park, N.J., under the trade designation “STYROFLEX”, from Kraton Polymers, Houston, Tex., under the trade designation “KRATON”, from Dow Chemical, Midland, Mich., under the trade designation “PELLETHANE”, “ENGAGE”, “INFUSE”, VERSIFY?, or “NORDEL”, from DSM, Heerlen, Netherlands, under the trade designation “ARNITEL”, from E. I. duPont de Nemours and Company, Wilmington, Del., under the trade designation “HYTREL”, from ExxonMobil, Irving, Tex. under the trade designation “VISTAMAXX”, and more.

In some embodiments, polymeric materials used to make nettings described herein may comprise a colorant (e.g., pigment and/or dye) for functional (e.g., optical effects) and/or aesthetic purposes (e.g., each has different color/shade). Suitable colorants are those known in the art for use in various polymeric materials. Exemplary colors imparted by the colorant include white, black, red, pink, orange, yellow, green, aqua, purple, and blue. In some embodiments, it is desirable level to have a certain degree of opacity for one or more of the polymeric materials. The amount of colorant(s) to be used in specific embodiments can be readily determined by those skilled in the (e.g., to achieve desired color, tone, opacity, transmissivity, etc.). If desired, the polymeric materials may be formulated to have the same or different colors. When colored strands are of a relatively fine (e.g., less than 50 micrometers) diameter, the appearance of the web may have a shimmer reminiscent of silk.

In embodiments in which the polymeric ribbons and polymeric, oscillating strands are different colors (e.g., including different colorants), polymeric nettings can have unique, striking aesthetic appeal. Using different colors in the polymeric ribbons from the polymeric strands can result in an iridescence in which the color of the netting appears to be different depending upon the angle of viewing. Thus, in some embodiments, polymeric nettings according to the present disclosure have polymeric ribbons-like elements that are a different color from the polymeric, oscillating strands. As an example, the oscillating strand can be yellow and the straight, ribbon-like strand can be purple. The netting can, for example, initially appear as a single colored surface and a viewer observing the information layer from acute angle θ₁ can see a first color “A”. In some cases, for example, the first color “A” is yellow corresponding to the oscillating strand, but as the viewing angle is increased to normal and eventually obtuse to the netting, the color may change to a second color “B”, for example the purple corresponding to the straight strands. In some cases, the changes that can be seen in the image vary continuously as the viewing angle is increased from an acute angle with the surface toward the normal to the netting surface. In some cases, the changes can be more abrupt, and even stepwise changes in the image can occur. Without wishing to be bound by theory, the viewing angle dependent color may result from a difference in height between the alternating polymeric strands over the width of the netting surface, as well as the selection of complimentary or contrasting colors. A slightly higher purple oscillating strand, for example, may partially obscure the relatively shorter yellow straight strand at certain view angles, rendering the appearance of the netting demonstrably purple.

Strands made using methods described herein do not substantially cross over each other (i.e., at least 50 (at least 55, 60, 65, 70, 75, 80, 85, 90, 95, 99, or even 100) percent by number).

In some embodiments, nettings described herein have a thickness up to 750 micrometers (in some embodiments, up to 500 micrometers, 250 micrometers, 100 micrometers, 75 micrometers, 50 micrometers, or even up to 25 micrometers; in a range from 10 micrometers to 750 micrometers, 10 micrometers to 750 micrometers, 10 micrometers to 500 micrometers, 10 micrometers to 250 micrometers, 10 micrometers to 100 micrometers, 10 micrometers to 75 micrometers, 10 micrometers to 50 micrometers, or even 10 micrometers to 25 micrometers).

In some embodiments, the polymeric strands have an average width in a range from 10 micrometers to 500 micrometers (in a range from 10 micrometers to 400 micrometers, or even 10 micrometers to 250 micrometers).

In some embodiments, nettings described herein have a basis weight in a range from 5 g/m² to 900 g/m² (in some embodiments, 10 g/m² to 800 g/m², 10 g/m² to 600 g/m², 10 g/m² to 400 g/m² , or even 400 g/m² to 600 g/m²) for example, nettings as-made from dies described herein. In some embodiments, nettings described herein after being stretched have a basis weight in a range from 0.5 g/m² to 40 g/m² (in some embodiments, 1 g/m² to 20 g/m²).

In some embodiments, nettings described herein have a strand pitch in a range from 0.5 mm to 20 mm (in some embodiments, in a range from 0.5 mm to 10 mm).

It has been observed that when some of the embodiments of netting made according to the present disclosure are stretched, they will relax to a length that is less than their original length before stretching. While not wishing to be bound by theory, it is believed that this is due to curling of the bond regions within the netting structure.

Some embodiments of polymeric nettings described herein are particularly useful, for example, for breathable compression wraps (i.e., wraps having a moisture vapor transmission rate (MVTR) value of at least 500 g/m²/day as measured using ASTM E 96 (1980) at 40° C.: the use of this test in connection with web material is discussed in U.S. Pat. No. 5,614,310 (Delgado et al.), the disclosures of which are incorporated herein by reference). When wrapping a limb with a compression wrap, it is typical to apply the wrap so that one course partially overlaps the previous course. Typically therapeutic regimens performed with compression wraps apply a force in a range from about 14 to about 35 mm Hg to the wrapped portion of the patient's body (see, e.g., the discussion at, “Compression Bandaging in the Treatment of Venous Leg Ulcers;” S. Thomas; World Wide Wounds, September 1997). It is therefore convenient for a compression wrap to have some extensibility so that minor changes in the diameter of the patient's limbs will not drastically change the compression force against the skin from the target pressure prescribed for the patient's indication. The compression wrap force can be measured as described in “Is Compression Bandaging Accurate? The Routine Use of Interface Pressure Measurements in Compression Bandaging of Venous Leg Ulcers;” A. Satpathy, S. Hayes and S. Dodds; Phlebology 2006 21: 36, the disclosure of which is incorporated herein by reference. In some embodiments, nettings described herein are convenient for use as compression wrap, for example, have openings in each of the first and second major surfaces that comprise in a range from 10 to 75 percent of their respective surface areas.

Optionally, nettings described herein are attached to a backing. The backings may be, for example, one of a film, net, or non-woven. Films may be particularly desirable, for example, for applications utilizing clear printing or graphics. Nonwovens or nets may be particularly desirable, for example, where a softness and quietness that films typically do not have is desired. The netting may be stretched and bonded between at least two layers of film or nonwoven where the bond points have a plurality (at least two) of bond points that do not include the netting in the bond. Alternatively, an unstretched netting could be bonded between at least two layers of film or nonwoven where the bond points have a plurality (at least two) of bond points that do not include the netting in the bond. These constructions may require subsequent stretching, either localized (“ring rolling”) or global, to become an activated elastic laminate.

In some embodiments, nettings described herein are elastic. In some embodiments, the polymeric strands have a machine direction and a cross-machine direction, wherein the netting or arrays of polymeric strands is elastic in machine direction, and inelastic in the cross-machine direction. Elastic means that the material will substantially resume its original shape after being stretched (i.e., will sustain only small permanent set following deformation and relaxation which set is less than 50 percent (in some embodiments, less than 25, 20, or even less than 10 percent) of the original length at moderate elongation (i.e., about 400-500%; in some embodiments, up to 300% to 1200%, or even up to 600 to 800%) elongation at room temperature). The elastic material can be both pure elastomers and blends with an elastomerie phase or content that will still exhibit substantial elastomeric properties at room temperature.

It is within the scope of the instant disclosure to use heat-shrinkable and non-heat shrinkable elastics. Non-heat shrinkable means that the elastomer, when stretched, will substantially recover sustaining only a small permanent set as discussed above at room temperature (Le., about 25° C.).

In some embodiments, nettings described herein of alternating first and second polymeric strands exhibit at least one of diamond-shaped or hexagonal-shaped openings.

In some embodiments, the polymeric strands have an average width in a range from 10 micrometers to 500 micrometers (in a range from 10 micrometers to 400 micrometers, or even 10 micrometers to 250 micrometers).

In some embodiments, the strands (i.e., the first strands, second strands, and bond regions, and other optional strands, each have thicknesses that are substantially the same.

In some embodiments, the bond regions have an average largest dimension perpendicular to the strand thickness, and wherein the average largest dimension of the bond regions is at least 2 (in some embodiments, at least 3, 4, 5, 10, or even at least 15) times greater than the average width of at least one of the first strands or the second strands.

In some embodiments, netting described herein includes an array of engagement posts (e.g., hooks) for engaging with the netting. Engagement hooks can be made as is known in the art (see, for example, U.S. Pat. No 5,077,870 (Melbye et al.)).

Nettings of polymeric strands described herein have a variety of uses, including wound care and other medical applications (e.g., elastic bandage-like material, surface layer for surgical drapes and gowns, and cast padding), tapes (including for medical applications), filtration, absorbent articles (e.g., diapers and feminine hygiene products) (e.g., as a layer(s) within the articles and/or as part of an attachment system for the articles or elastic components), pest control articles (e.g., mosquito nettings), geotextile applications (e.g., erosion control textiles), water/vapor management in clothing, reinforcement for nonwoven articles (e.g., paper towels), self bulking articles (e.g., for packaging) where the netting thickness is increased by stretching nettings with first strands have average first yield strength, and wherein the second strands have an average second yield strength that is different (e.g., at least 10 percent different) than the first yield strength, floor coverings (e.g., rugs and temporary mats), grip supports for tools, athletic articles, breathable elastic wrist and headbands, pattern coated adhesives, and pattern coated adhesives.

Advantages of some embodiments of nettings described herein when used as a backing, for example, for some tapes and wound dressings can include conformability, particularly in the cross direction (e.g., at least 50% elongation in the machine direction).

In some embodiments, nettings described herein are made of, or coated with, hydrophilic material to enhance moisture management. In some embodiments, nettings described herein are useful to manage wound moisture by transporting excess exudate from wounds, and in some embodiments, nettings described herein are made of bioresorbable polymers.

In some filtration applications, the netting can be used, for example, to provide spacers between filtering layers for filtration packs and/or to provide rigidity and support for filtration media. In some embodiments, several layers of the netting are used, where each layer is set to provide optimal filtering. Also, in some embodiments, the elastic feature of some nettings described herein can facilitate expansion the filter as the filter fills up.

In some embodiments, nettings described herein have high and low modulus strands such that stretching netting having a curled bond area can generate a lofted, accessible fiber for hook attachment (i.e., for an attachment system). In such oriented nettings attachment loops can have fiber strengths that are greater than unoriented nettings.

In some embodiments, nettings described herein that are elastic can flex in the machine direction, cross direction, or both directions, which can provide, for example, comfort and fit for diapers and the like. Elastic netting can also provide a breathable, soft, and flexible attachment mechanism (e.g., elastic netting can be attached to posts that fit through the elastic net, the elastic netting can be made with a ribbon region section attached to the netting to provide the fingerlift, the elastic can be made as elastic in one direction and inelastic in the second direction with an elastic and inelastic strand, or the ribbon region section can have molded hooks to provide attachment to a loop).

In some embodiments, nettings described herein useful as grip supports for tools, athletic articles, etc. are made using high friction polymers.

Some embodiments of nettings described herein can be used as or in disposable absorbent articles that may be useful, for example as personal absorbent articles for managing bodily fluids (e.g., perspiration, urine, blood, and menses) and disposable household wipes used to clean up similar fluids or typical household spills.

Advantages and embodiments of this invention are further illustrated by the following examples, but the particular materials and amounts thereof recited in these examples, as well as other conditions and details, should not be construed to unduly limit this invention. All parts and percentages are by weight unless otherwise indicated.

### EXAMPLE 1

A coextruded net was produced using the die configuration generally depicted in FIG. 14 and assembled with the multi shim repeating pattern of extrusion orifices as generally illustrated in FIG. 10. The thickness of the shims in the repeat sequence was 4 mils (0.102 mm) for shims 4440 and 4540. The thickness of the shims in was 8 mils (0.032 mm) for shims 4640. The height of dispensing orifices of 4566 and 4666 were each cut to 15 mils (0.381 mm). The extrusion orifices were aligned in a collinear, alternating arrangement, and resulting dispensing surface was as shown in FIG. 10. As assembled the width of the oscillating dispensing opening 6566 was 0.203 mm, the width of the straight dispensing opening 6666 was 0.406mm, and the land spacings between openings were 0.203 mm. The total width of the shim setup was about 12.5 cm.

The inlet fittings on the two end blocks were each connected to two conventional single-screw extruders. Extruder feeding cavities A and B were loaded with styrene-ethylene/butylene-styrene block copolymer elastomer (obtained under the trade designation “G1645 M” from Kraton, Belpre, Ohio) and dry blended with 3% blue or yellow colorant masterbatch resin, (blue colorant obtained under the trade designation “BLUE IN ELASTOMER (KIC-11)” from Clariant, Minneapolis, Minn., yellow obtained under the trade designation “FUTURO 116 YELLOW” from Americhem, Cuyahoga Falls, Ohio).

The flow rate of the polymer exiting the both dispensing openings 6566 and 6666 was 2.72 kg/hr. The melt was extruded vertically into an extrusion quench takeaway. The quench takeaway speed was 2.74 m/min, and the melt drop distance was 2 cm. The extrusion temperature was 260° C. The blue polymer was oscillating, exiting opening 6566. The quench roll was a smooth, temperature-controlled chrome plated 20-cm diameter steel roll. The quench temperature, which was 10° C., was controlled with internal water flow. The web was further cooled on the quench roll with compressed air flow through four 2.5-inch (6.35 cm) Loc-Line ® Swivel Nozzle 75 (Lockwood Products, INC, Lake Oswego, Oreg.). The web path wrapped 180 degrees around the chrome steel roll and then to a windup roll.

Using an optical microscope, at 50× magnification, the dimensions of the resulting polymeric net were determined as identified below. The height of the elements in the A and B direction are measured from the center point of the straight, ribbon-like strand. The center point of the straight, ribbon-like strand was determined with the radius function in the Keyence VMX-100 software. The center was determined by creating a circle where the top and bottom of the circle's circumference where placed at the top and bottom of the straight strand. The basis weight of the polymeric netting was found to be 268 g/m², and its overall caliper was 5.01 mm.

| | |
|---|---|
| Oscillating strand width: | 272 μm |
| Oscillating strand height: Side A | 259.2 μm |
| Oscillating strand height: Side B | 228.8 μm |
| Straight strand width: | 396.7 μm |
| Straight strand height: Side A | 201.3 μm |
| Straight strand height: Side B | 201.4 μm |

### EXAMPLE 2

A co-extrusion die was assembled with a multi shim repeating pattern of extrusion orifices as illustrated in FIG. 11A. The thickness of the all individual shims in the repeat sequence was 4 mils (0.102 mm). The height of dispensing orifices 4666 of shims 4640 were cut to 15 mils (0.381 mm). The height of dispensing orifices 4766 of shims 4740 were cut to 20 mils (0.508 mm). The extrusion orifices were aligned in a collinear, alternating arrangement, and resulting dispensing surface was as shown in FIG. 11B. The total width of the shim setup was about 15 cm.

The inlet fittings on the two end blocks were each connected to two conventional single-screw extruders. Each extruder feeding cavities A and B were loaded with styrene-ethylene/butylene-styrene block copolymer elastomer (a 7:1 dry blend of pellets, both obtained from Kraton, Belpre, Ohio under the trade designation “113012J”: “113012E”) and then dry blended with 2 wt % colorant masterbatch resin, (green colorant obtained under the trade designation “EMERALD 17-5641” from Americhem, Cuyahoga Falls, Ohio red obtained under the trade designation “RED IN ELASTOMER (KIC-11)” from Clariant, Minneapolis, Minn.)

The green polymer was oscillating, being extruded from dispensing opening 6766. The extruded polymer netting was quenched and cooled like Example 1. Other relevant process conditions are listed in below:

| | | |
|---|---|---|
| Orifice width for the first cavity: | 0.305 | mm |
| Orifice height for the first cavity: | 0.381 | mm |
| Orifice width of the second cavity: | 0.61 | mm |
| Orifice height of the second cavity: | 0.508 | mm |
| Land spacing between orifices | 0.305 | mm |
| Flow rate of oscillating polymer | 4.8 | kg/hr. |
| Flow rate of second polymer | 2.1 | kg/hr. |
| Extrusion temperature | 232° | C. |
| Quench roll temperature | 10° | C. |
| Quench takeaway speed | 2.83 | m/min. |
| Melt drop distance | 4 | cm |

Using an optical microscope, at 50× magnification, the dimensions of the resulting polymeric net were determined as identified in Table 2 below. The height of the elements in the A and B direction are measured from the center point of the straight strand. The center point of the straight strand was determined with the radius function in the Keyence VMX-100 software. The center was determined by creating a circle where the top and bottom of the circle's circumference where placed at the top and bottom of the straight strand. The basis weight of the polymeric netting was found to be 280 g/m², and its overall caliper was 4.478 mm.

| | |
|---|---|
| Oscillating strand width: | 564.7 μm |
| Oscillating strand height: Side A | 188.4 μm |
| Oscillating strand height: Side B | 231.8 μm |
| Straight strand width: | 441.6 μm |
| Straight strand height: Side A | 147 μm |
| Straight strand height: Side B | 153.5 μm |

### EXAMPLE 3

A co-extrusion die was assembled with a multi shim repeating pattern of extrusion orifices as illustrated in FIG. 12A. The thickness of the shims in the repeat sequence was 4 mils (0.102 mm) for shims 4840. The thickness of the shims in the repeat sequence was 8 mils (0.203 mm) for shims 4440. The thickness of the shims in the repeat sequence was 16 mils (0.406 mm) for shims 4940. The height of dispensing orifices of shims 4866 and 4966 were cut to 30 mils (0.762 mm) The extrusion orifices were aligned in a collinear, alternating arrangement, and resulting dispensing surface was as shown in FIG. 12B. The total width of the shim setup was about 15 cm.

The inlet fittings on the two end blocks were each connected to two conventional single-screw extruders. Each extruder feeding cavities A and B were loaded with styrene-ethylene/butylene-styrene block copolymer elastomer (a 7:1 dry blend of pellets, both obtained from Kraton, Belpre, Ohio under the trade designation “113012H”:“113012E”) and then dry blended with 2 wt % colorant masterbatch resin, (yellow colorant obtained under the trade designation “FUTURO 116 YELLOW” from Americhem, Cuyahoga Falls, Ohio red obtained under the trade designation “RED IN ELASTOMER (KIC-11)” from Clariant, Minneapolis, Minn.)

The yellow polymer was oscillating and emerged from opening 6860. The extruded polymer netting was quenched and cooled as in Examples 1 and 2. Other relevant process conditions are listed in below:

| | | |
|---|---|---|
| Orifice width for the first cavity: | 0.406 | mm |
| Orifice height for the first cavity: | 0.762 | mm |
| Orifice width of the second cavity: | 0.813 | mm |
| Orifice height of the second cavity: | 0.762 | mm |
| Land spacing between orifices | 0.406 | mm |
| Flow rate of first, oscillating polymer | 4.3 | kg/hr. |
| Flow rate of second polymer | 2.1 | kg/hr. |
| Extrusion temperature | 232° | C. |
| Quench roll temperature | 10° | C. |
| Quench takeaway speed | 1.98 | m/min. |
| Melt drop distance | 4 | cm |

Using an optical microscope, at 50× magnification, the dimensions of the resulting polymeric net were determined as listed below. The height of the elements in the A and B direction are measured from the center point of the straight strand and are shown below. The basis weight of the polymeric netting was found to be 435 g/m², and its overall caliper was 8.983 mm.

| | |
|---|---|
| Oscillating strand width: | 558.8 μm |
| Oscillating strand height: Side A | 407 μm |
| Oscillating strand height: Side B | 488 μm |
| Straight strand width: | 557.4 μm |
| Straight strand height: Side A | 234.4 μm |
| Straight strand height: Side B | 240.7 μm |

### EXAMPLE 4-7 AND COMPARATIVE EXAMPLES 1 AND 2

A co-extrusion die was assembled with a multi shim repeating pattern of extrusion orifices as illustrated in FIG. 12A. The thickness of the shims in the repeat sequence was 4 mils (0.102 mm) for shims 4840. The thickness of the shims in the repeat sequence was 8 mils (0.203 mm) for shims 4440. The thickness of the shims in the repeat sequence was 16 mils (0.406 mm) for shims 4940. The height of dispensing orifices of shims 4866 and 4966 were cut to 30 mils (0.762 mm). The extrusion orifices were aligned in a collinear, alternating arrangement, and resulting dispensing surface was as shown in FIG. 12B. The total width of the shim setup was about 15 cm.

The inlet fittings on the two end blocks were each connected to two conventional single-screw extruders. Each extruder feeding cavities A and B were loaded with styrene-ethylene/butylene-styrene block copolymer elastomer (obtained under the trade designation “G1645 M” from Kraton Polymers, Belpre, Ohio) and then dry blended with 2 wt % colorant masterbatch resin, (yellow colorant obtained under the trade designation “FUTURO 116 YELLOW” from Americhem, Cuyahoga Falls, Ohio and purple colorant obtained under the trade designation “PAN266C BLUE” from Clariant, Minneapolis, Minn.) The extruded polymer netting was quenched and cooled as in Examples 1-3. Other process conditions are listed in Table 1 below:

TABLE 1
Exemplary process conditions

| | Compar- | Compar- | | | | |
|---|---|---|---|---|---|---|
| | ative | ative | | | | |
| | Example 1 | Example 2 | Exam- | Exam- | Exam- | Exam- |
| | (CE1) | (CE2) | ple 6 | ple 7 | ple 8 | ple 9 |
| Oscillating | Yellow | Purple | Yellow | Purple | Yellow | Purple |
| Strand | | | | | | |
| Color | | | | | | |
| Straight | Yellow | Purple | Purple | Yellow | Purple | Yellow |
| Strand | | | | | | |
| Color | | | | | | |
| Flow | 2.72 | 2.72 | 2.72 | 2.72 | 3.86 | 3.86 |
| rate - | | | | | | |
| 1^{st} | | | | | | |
| polymer | | | | | | |
| (kg/hr) | | | | | | |
| Flow | 2.72 | 2.72 | 2.72 | 2.72 | 1.59 | 1.59 |
| rate - | | | | | | |
| 2^{nd} | | | | | | |
| polymer | | | | | | |

The following process conditions were shared among CE1, CE2 and Examples 4-7:

| | | |
|---|---|---|
| Orifice width for the first cavity: | 0.406 | mm |
| Orifice height for the first cavity: | 0.762 | mm |
| Orifice width of the second cavity: | 0.813 | mm |
| Orifice height of the second cavity: | 0.762 | mm |
| Land spacing between orifices | 0.406 | mm |
| Extrusion temperature | 260° | C. |
| Quench roll temperature | 10° | C. |
| Quench takeaway speed | 1.83 | m/min. |
| Melt drop distance | 4 | cm |

Using an optical microscope, at 50× magnification, the dimensions of the resulting polymeric net were determined as identified in Table 2 below. The height of the elements in the A and B direction are measured from the center point of the straight strand. The center point of the straight strand was determined with the radius function in the Keyence VMX-100 software. The center was determined by creating a circle where the top and bottom of the circle's circumference where placed at the top and bottom of the straight strand. The basis weight of the polymeric netting and overall caliper were measured as well.

TABLE 2
Netting dimensions

| | CE1 | CE2 | Ex. 4 | Ex. 5 | Ex. 6 | Ex. 7 |
|---|---|---|---|---|---|---|
| Net basis | 391 | 398 | 398 | 402 | 402 | 429 |
| weight | | | | | | |
| (gsm) | | | | | | |
| Overall Net | 812.3 | 825.4 | 839.9 | 842.9 | 838.4 | 828.9 |
| Caliper | | | | | | |
| Oscillating | 504 | 490.9 | 493.7 | 494.3 | 778.2 | 789 |
| Strand | | | | | | |
| width (μm) | | | | | | |
| Oscillating | 393.9 | 373.6 | 440.1 | 407.4 | 448.1 | 442.5 |
| Strand | | | | | | |
| height: Side | | | | | | |
| A (μm) | | | | | | |
| Oscillating | 388.1 | 449 | 401.2 | 430.2 | 333.1 | 430.1 |
| Strand | | | | | | |
| height: Side | | | | | | |
| B (μm) | | | | | | |
| Straight | 632.7 | 660.2 | 660.2 | 644.2 | 515.4 | 502.4 |
| Strand | | | | | | |
| width (μm) | | | | | | |
| Straight | 294.1 | 317.2 | 307 | 304.9 | 208.6 | 215.9 |
| Strand | | | | | | |
| height: Side | | | | | | |
| A (μm) | | | | | | |
| Straight | 311.5 | 312.8 | 304.2 | 313.5 | 208.6 | 218.8 |
| Strand | | | | | | |
| height: Side | | | | | | |
| B (μm) | | | | | | |

### Test Methods

### Maximum Hue Angle Change

To capture the change in perceptible color according to a changing viewing angle of the Exemplary samples, the rate of change of hue angle with view angle (|dhab/dqx|) was calculated every 2° from x-theta=−76° to x-theta=76° (described in more detail below). Samples were placed on a planar surface so that the individual sample lies flat. This planar surface will be referred to as the sample plane. Direction 1 is defined as the direction normal to the sample plane pointing out from the side the sample is on. Direction 2 is the down web direction and is parallel to the straight strand. The plane of reflection is parallel to Direction 1 and Direction 2. Light is incident on the sample from Direction 3, which is parallel to the plan of reflection and makes an angle θi with Direction 1. The viewing plane is perpendicular to Direction 2 and parallel to Direction 1. The sample is viewed from Direction 4, which is parallel to the viewing plane and makes an angle |θx| with Direction 1. The viewing angle Ox can range from −90° to 90° but most instruments can only measure out to about ±80°. A positive Ox signifies a direction that is in the same quadrant as Direction 3. A negative Ox signifies a direction that is in the other quadrant.

Color BRDFs (bidirectional reflectance distribution functions) were measured using a Radiant Zemax IS-SA™ (Imaging Sphere for Scatter and Appearance, available from Radiant Zemax, Redmond, Wash.) for each sample for incidence angles (α) of 20°, 40°, and 60°. In a Cartesian x-y-z coordinate system, the incidence angle α is the angle that the incident beam makes with the normal (i.e., z-axis) to the x-y plane of the sample, and the incidence angle α is located in the y-z plane (i.e., x=0). The CIE-X, CIE-Y and CIE-Z BRDF data was exported from the Radiant Zemax IS-SA software into CSV files using settings for Integration diameter=2°, Inclination resolution=2°, and Azimuthal resolution=2°. A MATLAB program was written to read the CSV files and calculate the CIE u′, v′, L*, a* and b* for each data point in the CSV file as described below:

CIE L*, a* and b*

The CIE L*, a*, b* and E* are calculated using the following relationships:

$$\begin{matrix}{a}^{*} = 500 * ( F ( X / Xn ) - F ( Y / Yn ) ) \\ {b}^{*} = 200 * ( F ( Y / Yn ) - F ( Z / Zn ) ) \\ {L}^{*} = 116 * F ( Y / Yn ) - 16\end{matrix}$$

$$F ( s ) = ( \begin{matrix}s \leq 0.008856 & 7.787 s + \frac{16}{116} \\ s > 0.008856 & {s}^{1 / 3}\end{matrix} )$$

Xn, Yn and Zn are CIE X, Y and Z values for the reference white.

For the Imaging Sphere, the default white point used is the CIE illuminant E point where Xn=Yn=Zn=⅓.

CIE Chroma (C*ab) and Hue Angle (h*ab)

$${C}_{ab}^{*} = \sqrt{{( Δ {a}^{*} )}^{2} + {( Δ {b}^{*} )}^{2}}$$

$${h}_{ab}^{*} = arctan ( \frac{{b}^{*}}{{a}^{*}} ) = atan 2 ( {b}^{*} , {a}^{*} )$$

To calculate the arctangent, MATLAB program was created to four quadrant inverse tangent function atan 2(y,x) was used, which give results ranging from −180° to 180°.

CIE u′ and IT′

The CIE u′, v′ and delta u′v′ were calculated using the following relationships:

$${u}^{′} = \frac{4 X}{X + 15 Y + 3 Z} {v}^{′} = \frac{9 Y}{X + 15 Y + 3 Z}$$

Using the CIE u′, v′, L*, a* and b* as calculated above, the color of the sample was evaluated by measuring the CIE hue angle h*ab.

$${h}_{ab}^{*} = arctan ( \frac{{b}^{*}}{{a}^{*}} ) = atan 2 ( {b}^{*} , {a}^{*} )$$

To calculate the arctangent, a MATLAB program was created to solve for the four quadrant inverse tangent function atan 2(y,x), which give results ranging from −180° to 180°.

h*_{ab}(θx) is the CIE hue angle of the sample when viewed from Direction 4.

The hue angle change is defined as

$$Δ {h}_{ab}^{*} ( {θ}_{x} ) = \{ \begin{matrix}\vert {h}_{ab}^{*} ( {θ}_{x} ) - {h}_{ab}^{*} ( 0 ) \vert & \vert {h}_{ab}^{*} ( {θ}_{x} ) - {h}_{ab}^{*} ( 0 ) \vert \leq 180 \\ 360 - \vert {h}_{ab}^{*} ( {θ}_{x} ) - {h}_{ab}^{*} ( 0 ) \vert & \vert {h}_{ab}^{*} ( {θ}_{x} ) - {h}_{ab}^{*} ( 0 ) \vert > 180\end{matrix} \}$$

The rate of change of hue angle with view angle (|dhab/dqx|) at a given x-theta (θₓ) is defined by the following relationship.

$$\vert \frac{dhab}{dqx} \vert = \vert \frac{Δ hab}{Δθ x} \vert = \vert \frac{hab ( {θ}_{x} + \frac{Δθ x}{2} ) - hab ( {θ}_{x} - \frac{Δθ x}{2} )}{Δθ x} \vert$$

where Δθₓ=4° is used. This rate is calculated at every two degrees in the interval θx=[−76°, 76°] with θy=0. The delta hue angle magnitude is the absolute value of the change in hue angle from θx=0. A maximum hue angle at any given θx above 5 indicates an observable change in color. A maximum hue angle at any given θx above 15 indicates a dramatic change in color.

In addition, color was also measured for the total integrated reflected light. In this case, a* and b* are calculated from the total integrated CIE X, Y and Z, or θi/Diffuse.

In addition, the measurement is made with the sample rotated 90° so that Direction 2 is in the cross web direction, the direction perpendicular to the straight strands.

The total hue angle change from θi=θ1 to θ2 is defined as

$$Δ {h}_{ab}^{*} ( {θ}_{1} to {θ}_{2} / diffuse ) = \{ \begin{matrix}\vert \begin{matrix}{h}_{ab}^{*} ( {θ}_{2} / diffuse ) - \\ {h}_{ab}^{*} ( {θ}_{1} / diffuse )\end{matrix} \vert & \vert \begin{matrix}{h}_{ab}^{*} ( {θ}_{2} / diffuse ) - \\ {h}_{ab}^{*} ( {θ}_{1} / diffuse )\end{matrix} \vert \leq 180 \\ 360 - \vert \begin{matrix}{h}_{ab}^{*} ( {θ}_{2} / diffuse ) - \\ {h}_{ab}^{*} ( {θ}_{1} / diffuse )\end{matrix} \vert & \vert \begin{matrix}{h}_{ab}^{*} ( {θ}_{2} / diffuse ) - \\ {h}_{ab}^{*} ( {θ}_{1} / diffuse )\end{matrix} \vert > 180\end{matrix} \}$$

If θi/Diffuse is below 3, the difference in color (if any) as a function of viewing angle may be less perceptible and/or susceptible to measurement error. In other embodiments, however, the hue angle shift may be noticeable at a θi/Diffuse of about 2.

Results

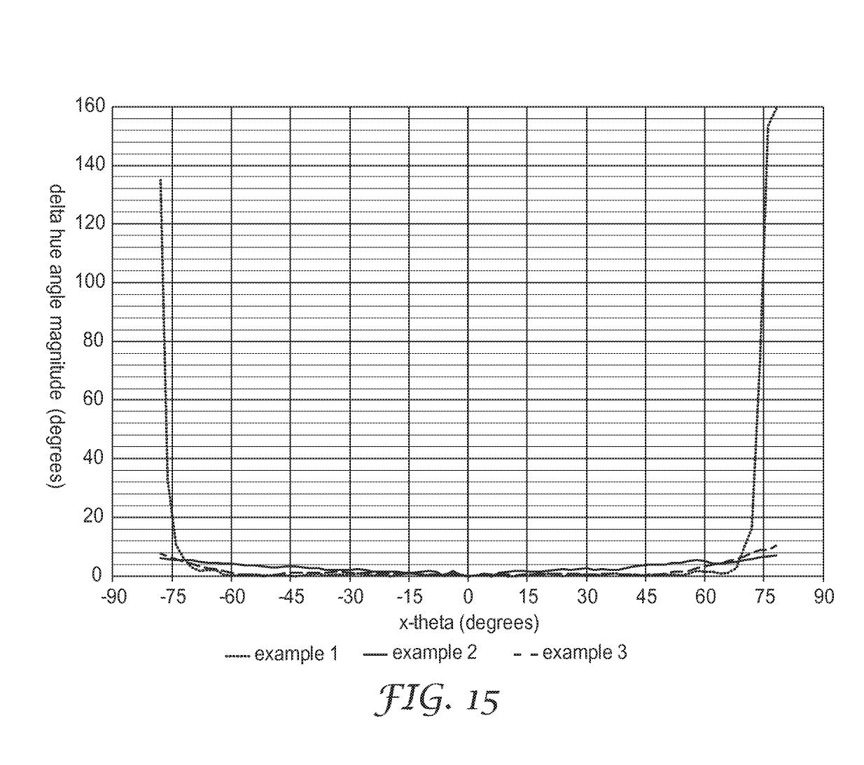

The rate of change of hue angle with view angle (|dhab/dqx|) was calculated every 2° from x-theta=−76° to x-theta=76° for Examples 1-3. The results are graphically represented in FIG. 15 and reproduced in Table 4 below.

TABLE 4
(|dhab/dqx|) at a given (θₓ)

| Example | −78 | −70 | −56 | −40 | −24 | 0 | 24 | 40 | 56 | 70 | 78 |
|---|---|---|---|---|---|---|---|---|---|---|---|
| example 1 | 134.9 | 3.0 | 0.6 | 0.5 | 0.3 | 0.0 | 0.0 | 0.3 | 0.9 | 9.9 | 159.4 |
| example 2 | 5.9 | 5.4 | 3.6 | 2.8 | 1.5 | 0.0 | 2.3 | 2.8 | 5.2 | 5.4 | 7.1 |
| example 3 | 7.7 | 4.3 | 0.4 | 1.2 | 1.1 | 0.0 | 0.8 | 0.6 | 1.8 | 6.7 | 10.3 |

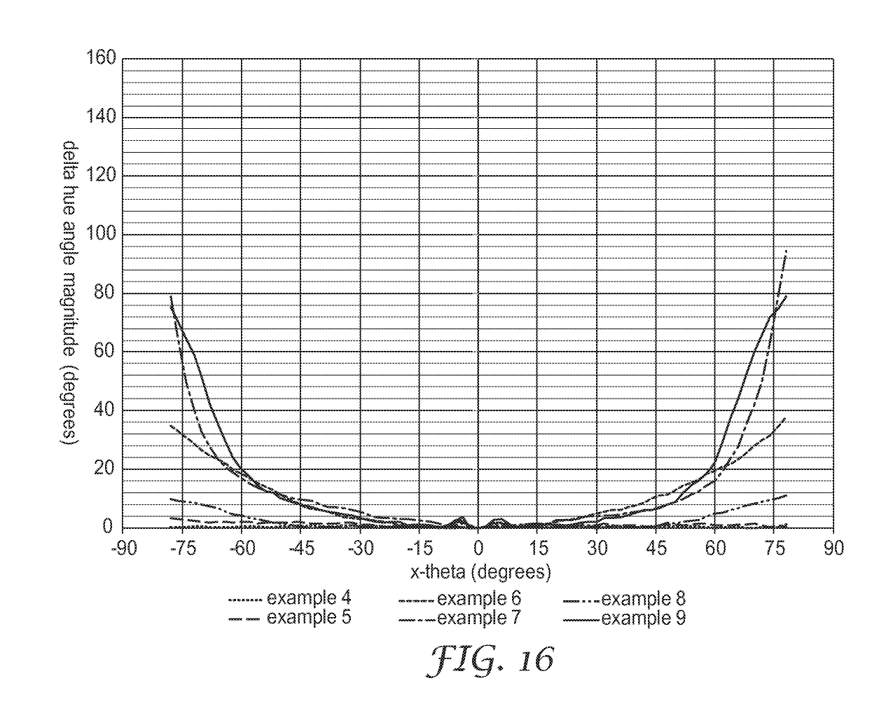

The rate of change of hue angle with view angle was calculated every 2° from x-theta=−76° to x-theta=76° for CE1, CE2 and Examples 4-7. The results are graphically represented in FIG. 16 and reproduced in Table 5 below. Note that examples CE1 and CE2 are identified as Examples 4 and 5 in FIG. 16, while Examples 4-7 are identified as Examples 6-9.

TABLE 5
(|dhab/dqx|) at a given (θₓ)

| Example | −78 | −70 | −56 | −40 | −24 | 0 | 24 | 40 | 56 | 70 | 78 |
|---|---|---|---|---|---|---|---|---|---|---|---|
| CE 1 | 0.2 | 0.4 | 0.5 | 0.7 | 0.2 | 0.0 | 0.1 | 0.1 | 0.5 | 0.0 | 1.2 |
| CE 2 | 3.4 | 2.0 | 1.6 | 1.4 | 0.5 | 0.0 | 0.3 | 0.7 | 1.5 | 1.3 | 0.3 |
| example 4 | 34.8 | 26.5 | 15.4 | 6.0 | 2.1 | 0.0 | 2.7 | 7.7 | 16.4 | 27.9 | 37.8 |
| example 5 | 79.1 | 32.7 | 13.9 | 8.1 | 3.4 | 0.0 | 3.0 | 5.5 | 12.6 | 42.1 | 94.3 |
| example 6 | 9.8 | 7.9 | 3.0 | 0.1 | 0.1 | 0.0 | 1.0 | 0.1 | 2.7 | 8.4 | 11.0 |
| example 7 | 75.4 | 51.4 | 14.7 | 6.1 | 1.8 | 0.0 | 1.2 | 5.0 | 16.2 | 60.3 | 79.1 |

The greater the maximum hue angle change for a given set of viewing angles, the more likely the change will be perceived. Comparative examples 1 and 2 exhibit relatively little change in perceptible color over the range of viewing angles, as evidenced by a maximum hue angle changes of less than 4. Without wishing to be bound by theory, the combination of two or more complimentary colors and a distinct disparity between straight and oscillating strand heights result in a more striking shift in color and aesthetic appeal.

### Embodiments

A. A netting comprising an array of polymeric strands periodically joined together at bond regions throughout the array, and wherein the array comprises a first polymer having a first color and a second polymer having a second color, and wherein the first color is different than the second color.

B. The netting of embodiment A, wherein the netting has a thickness of at least 10 micrometers and no greater than 750 micrometers.

C. The netting of embodiment A, wherein the netting has a basis weight of at least 10 g/m² and no greater than 600 g/m².

D. The netting of embodiment A, wherein the polymeric strands do not substantially cross over each other within the array.

E. The netting of any of previous embodiments, wherein the first polymer includes a first colorant and wherein the second polymer includes a second colorant.

F. The netting of any of the previous embodiments, wherein the first color or second color is selected from the group consisting of white, black, red, pink, orange, yellow, green, aqua, purple, and blue.

G. The netting of any of the previous embodiments, wherein array includes a pattern of alternating first and second strands, the first polymer defining a first alternating strand and the second polymer defining a second alternating strand.

H. The netting of any of the previous embodiments, wherein the first polymer is different than the second polymer.

I. An elastic bandage comprising the netting of any of the preceding embodiments.

J. A compression product comprising the netting of any of embodiments A-G.

K. A netting comprising an array of polymeric strands periodically joined together at bond regions throughout the array, and wherein the array comprises a first polymer having a first color and a second polymer having a second color, and wherein the first color is different than the second color, and wherein the netting exhibits the first color at a first viewing angle and the second color at a second viewing angle.

L. The netting of embodiment K, wherein the array comprises polymeric ribbons and polymeric strands, wherein the polymeric ribbons have a major surface that is intermittently bonded to only one polymeric strand at a bond region, wherein the polymeric ribbons comprise the first polymer and the polymeric strands comprise the second polymer, and wherein the first color is different than the second color.

M. The netting of embodiment K, wherein the polymeric strands oscillate in a generally sinusoidal pattern between adjacent ribbons.

N. The netting of embodiments L or M wherein the oscillating strand includes a greater height from the center line than the ribbon.

O. The netting of embodiments L or M wherein the ribbon strand includes a greater height from the center line than the oscillating strand.

P. The netting of embodiment K, wherein the polymeric ribbon is substantially straight, and wherein a polymeric strand oscillates between adjacent polymeric ribbons.

Q. The netting of any of the preceding embodiments, wherein the netting exhibits a 40°/Diffuse CIE chroma of at least 3 and a maximum hue angle change of at least 5 degrees in the interval θx=[−78°, 78°] when θi=40°.

R. The netting of any of the preceding embodiments, wherein the netting exhibits a 40°/Diffuse CIE chroma of at least 3 and a maximum hue angle change of at least 8 degrees in the interval θx=[−78°, 78°] when θi=40°.

S. The netting of any of the preceding embodiments, wherein the netting exhibits a 40°/Diffuse CIE chroma of at least 3 and a maximum hue angle change of at least 15 degrees in the interval θx=[−78°, 78°] when θi=40°.

T. An elastic bandage including the netting of any of the preceding embodiments K-S.

U. A compression product including the netting of the preceding embodiments K-S.

V. An extrusion die having at least first and second cavities, a first passageway extending from the first cavity into a first dispensing orifice, and second and third passageways extending from the second cavity to a second dispensing orifice, the cavities and orifices defined by a plurality of shims.

Foreseeable modifications and alterations of this disclosure will be apparent to those skilled in the art without departing from the scope and spirit of this invention. This invention should not be restricted to the embodiments that are set forth in this application for illustrative purposes.

What is claimed is:

1. A netting comprising an array of polymeric strands periodically joined together at bond regions throughout the array, and wherein the array comprises a first polymer having a first color and a second polymer having a second color, and wherein the first color is different than the second color.

2. The netting of claim 1, wherein the netting has a thickness of at least 10 micrometers and no greater than 750 micrometers.

3. The netting of claim 1, wherein the netting has a basis weight of at least 10 g/m² and no greater than 600 g/m².

4. The netting of claim 1, wherein the polymeric strands do not substantially cross over each other within the array.

5. The netting of claim 1, wherein the first color or second color is selected from the group consisting of white, black, red, pink, orange, yellow, green, aqua, purple, and blue.

6. The netting of claim 1, wherein the array includes a pattern of alternating first and second strands, the first polymer defining a first alternating strand and the second polymer defining a second alternating strand.

7. A netting comprising an array of polymeric strands periodically joined together at bond regions throughout the array, and wherein the array comprises a first polymer having a first color and a second polymer having a second color, and wherein the first color is different than the second color, and wherein the netting exhibits the first color at a first viewing angle and the second color at a second viewing angle.

8. The netting of claim 7, wherein the array comprises polymeric ribbons and polymeric strands, wherein the polymeric ribbons have a major surface that is intermittently bonded to only one polymeric strand at a bond region, wherein the polymeric ribbons comprise the first polymer and the polymeric strands comprise the second polymer, and wherein the first color is different than the second color.

9. The netting of claim 8, wherein the polymeric strands oscillate in a generally sinusoidal pattern between adjacent ribbons.

10. The netting of claim 9, wherein the oscillating strand includes a greater height from the center line than the ribbon.

11. The netting of claim 9, wherein the ribbon strand includes a greater height from the center line than the oscillating strand.

12. The netting of claim 9, wherein the polymeric ribbon is substantially straight, and wherein a polymeric strand oscillates between adjacent polymeric ribbons.

13. The netting of claim 8, wherein the netting exhibits a 40°/Diffuse CIE chroma of at least 3 and a maximum hue angle change is at least 5 degrees in the interval θx=[−78°, 78°] when θi=40°.

14. The netting of claim 8, wherein the netting exhibits a 40°/Diffuse CIE chroma of at least 3 and a maximum hue angle change is at least 8 degrees in the interval θx=[−78°, 78°] when θi=40°.

15. The netting of claim 8, wherein the netting exhibits a 40°/Diffuse CIE chroma of at least 3 and a maximum hue angle change of at least 15 degrees in the interval θx=[−78°, 78°] when θi=40°.

16. An elastic bandage or compression product comprising the netting of claim 13.

17. The elastic bandage or compression product of claim 16, wherein the netting exhibits a 40°/Diffuse CIE chroma of at least 3 and a maximum hue angle change of at least 15 degrees in the interval θx=[−78°, 78°] when θi=40°.

18. The elastic bandage or compression product of claim 16, wherein the array comprises polymeric ribbons and polymeric strands, wherein the polymeric ribbons have a major surface that is intermittently bonded to only one polymeric strand at a bond region, wherein the polymeric ribbons comprise the first polymer and the polymeric strands comprise the second polymer, and wherein the first color is different than the second color.

19. The elastic bandage of claim 18, wherein the polymeric strands oscillate in a generally sinusoidal pattern between adjacent ribbons.

20. The elastic bandage of claim 19, wherein the ribbon strand includes a greater height from the center line than the oscillating strand.
